(19) United States
(12) Patent Application Publication
(10) Pub. No.: US 2024/0110225 A1
(43) Pub. Date: Apr. 4, 2024

(54) METHOD, SYSTEM, AND APPARATUS FOR ANALYZING AN ANALYTE OF A SINGLE CELL

(71) Applicant: Mission Bio, Inc., South San Francisco, CA (US)

(72) Inventors: Aik Ooi, San Mateo, CA (US); David Ruff, San Francisco, CA (US); Saurabh Parikh, San Francisco, CA (US); Prithvi Singh, San Francisco, CA (US); Dalia Dhingra, San Francisco, CA (US)

(21) Appl. No.: 18/535,441

(22) Filed: Dec. 11, 2023

Related U.S. Application Data
(63) Continuation of application No. PCT/US22/32648, filed on Jun. 8, 2022.
(60) Provisional application No. 63/209,598, filed on Jun. 11, 2021.
(60) Provisional application No. 63/317,369, filed on Mar. 7, 2022.
(60) Provisional application No. 63/327,964, filed on Apr. 6, 2022.

(51) Int. Cl. C12Q 1/6806; C12Q 1/6827; C12Q 1/6844

(52) U.S. Cl. CPC C12Q 1/6806; C12Q 1/6827; C12Q 1/6844

(57) ABSTRACT

Disclosed herein are methods for analyzing one or more analytes of a cell by performing single-cell analysis. In one scenario, the one or more analytes are located on a surface of the cell. In one scenario, the one or more analytes are located internally within the cell. In one scenario, the one or more analytes include proteins located on a surface of the cell and located internally within the cell.

### CROSS REFERENCE TO RELATED APPLICATIONS

This application is a continuation of International (PCT) Patent Application No. PCT/US22/32648, filed Jun. 8, 2021, which claims the benefit of and priority to U.S. Provisional Patent Application No. 63/209,598, filed Jun. 11, 2021, U.S. Provisional Patent Application No. 63/317,369, filed Mar. 7, 2022, and U.S. Provisional Patent Application No. 63/327,964 filed, Apr. 6, 2022, the entire disclosures of each of which are hereby incorporated by reference in their entireties for all purposes.

### BACKGROUND

Proteins include a versatile group of macromolecules and can control cell division, metabolism and processes in a body, and thus can have an impact on the structure, function and regulation of the tissues and organs in the body. Protein analysis include experimental techniques for the detection, purification and identification of proteins, as well as the characterization of protein structure and function. Protein analysis methods may also involve analysis of protein of a cell, tissue, or organism under a specific, defined set of conditions. Therefore, it is valuable to interrogate protein expression of individual cells as it can reveal cellular states, examples of which include diseased states.

Current single cell analysis methods can analyze protein expression located on the surface of cells; however, currently, there are no available methods for analyzing expression of intracellular proteins.

### SUMMARY

Disclosed herein are methods, systems, and apparatuses to analyze one or more analytes of individual cells by performing single-cell analysis. Generally, the methods, systems, and apparatuses disclosed herein enable analyzing one or more analytes of single cells, such as analysis of surface proteins, intracellular proteins, RNA, and DNA, and/or analysis of genotypic factors (e.g., SNVs, indels, or CNVs) and phenotypic factors (e.g., proteins) from a number of (e.g., thousands of) individual cells. In various embodiments, the analyte of an individual cell refers to a protein of the cell. In various embodiments, the analyte of an individual cell refers to a protein located on a surface of the cell. In particular embodiments, the analyte of an individual cell refers to an intracellular protein located internally within the cell. In particular embodiments, the analyte of an individual cell refers to a protein located on a surface of the cell and an intracellular protein located internally within the cell. Surface proteins include cell membrane proteins, cell surface receptors, cytoplasmic proteins, phosphorylated proteins, epigenetics proteins and/or others that have implications in the processes (e.g., healthy or diseased processes) of individual cells. Intracellular proteins include cytoplasmic proteins, nuclear proteins, phosphorylated proteins, epigenetics proteins and/or others that have implications in the processes (e.g., healthy or diseased processes) of individual cells. In various embodiments, methods, systems, and apparatuses disclosed herein enable the analysis of surface and/or intracellular proteins, in addition to other cellular analytes (e.g., DNA and/or RNA of the cell).

Disclosed herein is a method for analyzing an analyte of a cell, the method comprising: obtaining a permeabilized cell; providing an antibody-oligonucleotide conjugate to the permeabilized cell, wherein the antibody-oligonucleotide conjugate enters the permeabilized cell to contact the analyte located internally within the cell to generate an intracellular antibody-oligonucleotide conjugate; performing a single-cell analysis of the cell, wherein performing the single-cell analysis comprises: encapsulating the permeabilized cell comprising the intracellular antibody-oligonucleotide conjugate in a droplet; lysing the permeabilized cell within the droplet to generate a cell lysate comprising the oligonucleotide or a complement of the oligonucleotide; optionally reverse cross-linking the cell lysate within the droplet using a reducing agent; re-encapsulating the cell lysate in a second droplet with reagents; generating amplicons from the oligonucleotide or the complement of the oligonucleotide by performing at least one reaction using the reagents; and sequencing the amplicons to determine presence or absence of the analyte of the permeabilized cell.

In some embodiments, obtaining a permeabilized cell comprises: fixing a cell using fixatives; quenching the fixatives; and permeabilizing and blocking the cell.

In some embodiments, providing an antibody-oligonucleotide conjugate to the permeabilized cell comprises: incubating the permeabilized cell with the antibody-oligonucleotide conjugate; and washing the permeabilized cell.

In some embodiments, providing an antibody-oligonucleotide conjugate to the permeabilized cell comprises: incubating the permeabilized cell with the antibody-oligonucleotide conjugate for 10 minutes to 30 hours.

In some embodiments, providing an antibody-oligonucleotide conjugate to the permeabilized cell comprises: incubating the permeabilized cell with the antibody-oligonucleotide conjugate for 10-25 hours.

In some embodiments, providing an antibody-oligonucleotide conjugate to the permeabilized cell comprises: incubating the permeabilized cell with the antibody-oligonucleotide conjugate for 16-20 hours.

In some embodiments, providing an antibody-oligonucleotide conjugate to the permeabilized cell comprises: incubating the permeabilized cell with the antibody-oligonucleotide conjugate overnight.

In some embodiments, providing an antibody-oligonucleotide conjugate to the permeabilized cell comprises: incubating the permeabilized cell with the antibody-oligonucleotide conjugate at a temperature between 0-25° C.

In some embodiments, providing an antibody-oligonucleotide conjugate to the permeabilized cell comprises: incubating the permeabilized cell with the antibody-oligonucleotide conjugate at a temperature between 2-8° C.

In some embodiments, providing an antibody-oligonucleotide conjugate to the permeabilized cell comprises: incubating the permeabilized cell with the antibody-oligonucleotide conjugate at a temperature between 3-6° C.

In some embodiments, providing an antibody-oligonucleotide conjugate to the permeabilized cell comprises: incubating the permeabilized cell with the antibody-oligonucleotide conjugate at a temperature of about 4° C.

In some embodiments, providing an antibody-oligonucleotide conjugate to the permeabilized cell comprises: washing the cell for at least 1 minute to wash away unbound antibody-oligonucleotide conjugates.

In some embodiments, providing an antibody-oligonucleotide conjugate to the permeabilized cell comprises: washing the permeabilized cell for at least 3 minutes to wash away unbound antibody-oligonucleotide conjugates.

In some embodiments, providing an antibody-oligonucleotide conjugate to the permeabilized cell comprises: washing the permeabilized cell for at least 5 minutes to wash away unbound antibody-oligonucleotide conjugates.

In some embodiments, providing an antibody-oligonucleotide conjugate to the permeabilized cell comprises: washing the permeabilized cell for one or more times to wash away unbound antibody-oligonucleotide conjugates.

In some embodiments, providing an antibody-oligonucleotide conjugate to the permeabilized cell comprises: washing the permeabilized cell for at least 2 times.

In some embodiments, providing an antibody-oligonucleotide conjugate to the permeabilized cell comprises: washing the permeabilized cell for at least 3 times.

In some embodiments, providing an antibody-oligonucleotide conjugate to the permeabilized cell comprises: washing the permeabilized cell for at least 4 times.

Additionally disclosed herein is a method for analyzing an analyte located on a surface of a cell and an analyte located internally within the cell, the method comprising: obtaining the cell comprising a surface antibody-oligonucleotide conjugate and an intracellular antibody-oligonucleotide conjugate, the surface antibody-oligonucleotide conjugate being generated by providing a first antibody-oligonucleotide conjugate to be bound to the analyte located on the surface of the cell, and the intracellular antibody-oligonucleotide conjugate being generated by permeabilizing the cell and providing a second antibody-oligonucleotide conjugate to enter the permeabilized cell to contact the analyte located internally within the cell; performing a single-cell analysis of the cell, wherein performing the single-cell analysis comprises: encapsulating the permeabilized cell comprising the surface antibody-oligonucleotide conjugate and the intracellular antibody-oligonucleotide conjugate in a first droplet; lysing the permeabilized cell within the first droplet to generate a cell lysate comprising a first oligonucleotide or a complement of the first oligonucleotide from the first antibody-oligonucleotide conjugate and a second oligonucleotide or a complement of the second oligonucleotide from the second antibody-oligonucleotide conjugate; optionally, reverse cross-linking the cell lysate within the first droplet using a reducing agent; re-encapsulating the cell lysate in a second droplet with reagents; generating first amplicons from the first oligonucleotide or the complement of the first oligonucleotide and second amplicons from the second oligonucleotide or the complement of the second oligonucleotide by performing at least one reaction using the reagents; and sequencing any one of the first and second amplicons to determine presence or absence of the analyte located on the surface of the cell and the analyte located internally within the permeabilized cell.

In some embodiments, obtaining the cell comprises: incubating the cell with at least one of the first and the second antibody-oligonucleotide conjugates; and washing the cell.

In some embodiments, incubating the cell with at least one of the first and the second antibody-oligonucleotide conjugates comprises: incubating the cell with at least one of the first and the second antibody-oligonucleotide conjugates for 10 minutes to 30 hours.

In some embodiments, incubating the cell with the first and the second antibody-oligonucleotide conjugates comprises: incubating the cell with the at least one of the first and the second antibody-oligonucleotide conjugates for 10-60 minutes.

In some embodiments, incubating the cell with at least one of the first and the second antibody-oligonucleotide conjugates comprises: incubating the cell with at least one of the first and the second antibody-oligonucleotide conjugates for 16-20 hours.

In some embodiments, incubating the cell with at least one of the first and the second antibody-oligonucleotide conjugates comprises: incubating the cell with at least one of the first and the second antibody-oligonucleotide conjugates overnight.

In some embodiments, incubating the cell with at least one of the first and the second antibody-oligonucleotide conjugates comprises: incubating the cell with at least one of the first and the second antibody-oligonucleotide conjugates at a temperature between 0 and 25° C.

In some embodiments, incubating the cell with at least one of the first and the second antibody-oligonucleotide conjugates comprises: incubating the cell with at least one of the first and the second antibody-oligonucleotide conjugates at a temperature between 2 and 25° C.

In some embodiments, incubating the cell with at least one of the first and the second antibody-oligonucleotide conjugates comprises: incubating the cell with at least one of the first and the second antibody-oligonucleotide conjugates on ice.

In some embodiments, incubating the cell with at least one of the first and the second antibody-oligonucleotide conjugates comprises: incubating the cell with at least one of the first and the second antibody-oligonucleotide conjugates at a temperature of about 4° C.

In some embodiments, washing the cell comprises: washing the cell for at least 1 minute to wash away unbound first antibody-oligonucleotide conjugates.

In some embodiments, washing the cell comprises: washing the cell for at least 3 minutes to wash away unbound first antibody-oligonucleotide conjugates.

In some embodiments, washing the cell comprises: washing the cell for at least 5 minutes to wash away unbound first antibody-oligonucleotide conjugates.

In some embodiments, washing the cell comprises: washing the cell for one or more times to wash away unbound first antibody-oligonucleotide conjugates.

In some embodiments, washing the cell comprises: washing the cell for at least 2 times.

In some embodiments, washing the cell comprises: washing the cell for at least 3 times.

In some embodiments, washing the cell comprises: washing the cell for at least 4 times.

In some embodiments, the method further comprises: providing one or more additional first antibody-oligonucleotide conjugates specific for one or more additional surface analytes to the cell.

In some embodiments, the one or more additional first antibody-oligonucleotide conjugates comprise two additional first antibody-oligonucleotide conjugates specific for two surface analytes.

In some embodiments, the one or more additional first antibody-oligonucleotide conjugates comprise three additional first antibody-oligonucleotide conjugates specific for three surface analytes.

In some embodiments, the one or more additional first antibody-oligonucleotide conjugates comprise four additional first antibody-oligonucleotide conjugates specific for four surface analytes.

In some embodiments, the one or more additional first antibody-oligonucleotide conjugates comprise five additional first antibody-oligonucleotide conjugates specific for five surface analytes.

In some embodiments, the one or more additional first antibody-oligonucleotide conjugates comprise forty-five additional first antibody-oligonucleotide conjugates specific for forty-five surface analytes.

In some embodiments, the method further comprises: providing one or more additional second antibody-oligonucleotide conjugates specific for one or more additional intracellular analytes to the permeabilized cell.

In some embodiments, the one or more additional second antibody-oligonucleotide conjugates comprise five additional second antibody-oligonucleotide conjugates specific for five intracellular analytes.

In some embodiments, the one or more additional second antibody-oligonucleotide conjugates comprise ten additional second antibody-oligonucleotide conjugates specific for ten intracellular analytes.

In some embodiments, the one or more additional second antibody-oligonucleotide conjugates comprise fifty additional second antibody-oligonucleotide conjugates specific for fifty intracellular analytes.

In some embodiments, the second antibody-oligonucleotide conjugate comprises a concentration of up to 13 nM per antibody.

In some embodiments, obtaining a cell comprises: fixing the cell using fixatives for at least 30 minutes.

In some embodiments, obtaining a cell comprises: fixing the cell using fixatives for at least 45 minutes.

In some embodiments, obtaining a cell comprises: fixing the cell using fixatives for at least 60 minutes.

In some embodiments, obtaining a cell comprises: fixing the cell using fixatives for at least 90 minutes.

In some embodiments, obtaining a cell comprises: fixing the cell using fixatives at a temperature between 4 and 50° C.

In some embodiments, obtaining a cell comprises: fixing the cell using fixatives at a temperature between 10 and 30° C.

In some embodiments, obtaining a cell comprises: fixing the cell using fixatives at a temperature between 20 and 25° C.

In some embodiments, obtaining a cell comprises: fixing the cell using 0.1 mM to 20 mM of one or more fixatives in a reactive volume using a background buffer.

In some embodiments, obtaining a cell comprises: fixing the cell using 0.5 mM to 10 mM of one or more fixatives in a reactive volume using a background buffer.

In some embodiments, obtaining a cell comprises: fixing the cell using 1 mM to 5 mM of one or more fixatives in a reactive volume using a background buffer.

In some embodiments, the reactive volume is from 0.01 to 10 mL.

In some embodiments, the reactive volume is from 0.05 to 5 mL.

In some embodiments, the reactive volume is from 0.1 to 1 mL.

In some embodiments, the background buffer is Dulbecco's phosphate-buffered saline (DPBS).

In some embodiments, obtaining a cell comprises: quenching the cell for at least 1 minute.

In some embodiments, obtaining a cell comprises: quenching the cell for at least 5 minutes.

In some embodiments, obtaining a cell comprises: quenching the cell for at least 10 minutes.

In some embodiments, obtaining a cell comprises: quenching the cell at a temperature between 10 and 50° C.

In some embodiments, obtaining a cell comprises: quenching the cell at a temperature between 10 and 30° C.

In some embodiments, obtaining a cell comprises: quenching the cell at a temperature between 20 and 25° C.

In some embodiments, obtaining a cell comprises: permeabilizing and blocking the cell for at least 10 minutes.

In some embodiments, obtaining a cell comprises: permeabilizing and blocking the cell for at least 20 minutes.

In some embodiments, obtaining a cell comprises: permeabilizing and blocking the cell for at least 30 minutes.

In some embodiments, obtaining a cell comprises: permeabilizing and blocking the cell at a temperature between 10 and 50° C.

In some embodiments, obtaining a cell comprises: permeabilizing and blocking the cell at a temperature between 10 and 30° C.

In some embodiments, obtaining a cell comprises: permeabilizing and blocking the cell at a temperature between 20 and 25° C.

In some embodiments, the single-cell analysis is performed for at least 1 million cells in one workflow.

In some embodiments, the reagents comprise one or more antibody tag primers.

In some embodiments, the one or more antibody tag primers comprises at least 10 primer reagents.

In some embodiments, the one or more antibody tag primers comprises at least 50 primer reagents.

In some embodiments, the one or more antibody tag primers comprises at least 100 primer reagents.

In some embodiments, the one or more antibody tag primers comprises at least 150 primer reagents.

In some embodiments, the reagents comprise one or more barcodes in the second droplet.

In some embodiments, the reagents comprise polymerase.

In some embodiments, the at least one reaction comprises nucleic acid amplification.

In some embodiments, the at least one reaction comprises polymerase chain reaction (PCR).

In some embodiments, the at least one reaction comprises loop-mediated isothermal amplification (LAMP).

In some embodiments, the cell lysate comprises genomic DNA.

In some embodiments, the method further comprises generating amplicons from the genomic DNA.

In some embodiments, the method further comprises sequencing the amplicons generated from the genomic DNA.

In some embodiments, further comprises determining presence or absence of one or more mutations based on the sequenced amplicons generated from the genomic DNA.

In some embodiments, the one or more mutations comprise any one of single-nucleotide polymorphism (SNV), insertion or deletion mutation (indel), or copy number variation (CNV).

In some embodiments, permeabilizing the cell comprises permeabilizing the cell using a permeabilization buffer, and wherein the permeabilization buffer comprises at least one of Triton™ X-100, Prionex® gelatin, salmon sperm DNA, mouse IgG, and EDTA.

In some embodiments, permeabilizing the cell comprises permeabilizing the cell using a permeabilization buffer, wherein the permeabilization buffer comprises a 0.1% solution.

In some embodiments, lysing the permeabilized cell within the droplet comprises: optionally applying extra reagents, wherein the amount of the extra reagents is less than 1 mM DTT.

In some embodiments, lysing the permeabilized cell within the droplet comprises: optionally applying extra reagents, wherein the amount of the extra reagents is less than 2 mM DTT.

In some embodiments, lysing the permeabilized cell within the droplet comprises: optionally applying extra reagents, wherein the amount of the extra reagents is less than 5M DTT.

In some embodiments, the method is applied to one or more cell lines.

In some embodiments, the analyte is at least one of surface protein, intracellular protein, or genomic DNA.

Additionally disclosed herein is a method for analyzing an analyte of a cell, the method comprising: obtaining a cell nucleus isolated from the cell; providing an antibody-oligonucleotide conjugate to the cell nucleus, wherein the antibody-oligonucleotide conjugate contacts the analyte of the cell nucleus to generate an intracellular antibody-oligonucleotide conjugate; performing a single-cell analysis of the cell, wherein performing the single-cell analysis comprises: encapsulating the cell nucleus comprising the intracellular antibody-oligonucleotide conjugate in a droplet; generating a cell nucleus lysate within the droplet comprising the oligonucleotide or a complement of the oligonucleotide; optionally reverse cross-linking the cell nucleus lysate within the droplet using a reducing agent; re-encapsulating the cell lysate in a second droplet with reagents; generating amplicons from the oligonucleotide or the complement of the oligonucleotide by performing at least one reaction using the reagents; and sequencing the amplicons to determine presence or absence of the analyte of the cell nucleus.

In some embodiments, obtaining the cell nucleus isolated from the cell comprises: incubating the cell nucleus with the antibody-oligonucleotide conjugate; and washing the cell nucleus.

In some embodiments, the method further comprises: providing one or more additional antibody-oligonucleotide conjugates specific for one or more additional analytes to the cell nucleus.

In some embodiments, the cell nucleus lysate comprises genomic DNA.

In some embodiments, the method further comprises generating amplicons from the genomic DNA.

In some embodiments, the method further comprises sequencing the amplicons generated from the genomic DNA.

In some embodiments, the method further comprises determining presence or absence of one or more mutations based on the sequenced amplicons generated from the genomic DNA.

In some embodiments, the one or more mutations comprise any one of single-nucleotide polymorphism (SNV), insertion or deletion mutation (indel), or copy number variation (CNV).

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A depicts an overall system environment including a single cell workflow device and a computational device for conducting single-cell analysis, in accordance with an embodiment.

FIG. 1B shows an embodiment of processing individual cells to generate amplicons for sequencing, in accordance with an embodiment.

FIG. 2A shows a flow process of performing a single cell analysis for analyzing one or more analytes of a cell, in accordance with an embodiment.

FIG. 2B shows a flow process of performing a single cell analysis for analyzing one or more analytes of a cell, in accordance with a first embodiment.

FIG. 2C shows a flow process of performing a single cell analysis for analyzing one or more analytes of a cell, in accordance with a second embodiment.

FIG. 2D shows a flow process of performing a single cell analysis for analyzing one or more analytes of a cell, in accordance with a third embodiment.

FIGS. 3A-3C show the steps of lysing and digesting in the first droplet as described in the step 165 in FIG. 1B, in accordance with an embodiment.

FIG. 4A illustrates the priming and barcoding of an antibody-conjugated oligonucleotide, in accordance with an embodiment.

FIG. 4B illustrates the priming and barcoding of genomic DNA, in accordance with an embodiment.

FIG. 5 depicts an example computing device for implementing system and methods described in reference to FIG. 1A.

FIGS. 6A-6C show example protocols for surface and intracellular protein workflows.

FIGS. 7A and 7B show example results obtained from the single cell analysis based on genomic DNA, intracellular proteins, and surface proteins.

FIGS. 8-16 show example results obtained from the single cell analysis based on genomic DNA, intracellular proteins, and/or surface proteins.

### DETAILED DESCRIPTION

### Definitions

Terms used in the claims and specification are defined as set forth below unless otherwise specified.

The term “about” refers to a ±10% variation from the nominal value unless otherwise indicated or inferred.

The term “subject” or “patient” are used interchangeably and encompass an organism, human or non-human, mammal or non-mammal, male or female.

The term “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “analyte” refers to a component of a cell. Cell analytes can be informative for understanding a state or behavior of a cell. Therefore, performing single-cell analysis of one or more analytes of a cell using the systems and methods described herein are informative for determining a state or behavior of a cell. Examples of an analyte include a nucleic acid (e.g., RNA, DNA, cDNA), a protein, a peptide, an antibody, an antibody fragment, a polysaccharide, a sugar, a lipid, a small molecule, or combinations thereof. In particular embodiments, a single-cell analysis involves analyzing protein analytes. In particular embodiments, a single-cell analysis involves analyzing surface protein analytes. In particular embodiments, a single-cell analysis involves analyzing intracellular protein analytes. In particular embodiments, a single-cell analysis involves analyzing two different analytes such as protein (e.g., intracellular anal; or surface protein) and DNA, protein (e.g., intracellular and/or surface protein) and RNA, or RNA and DNA. In particular embodiments, a single-cell analysis involves analyzing three or more different analytes of a cell, such as RNA, DNA, and protein.

The phrase “cell phenotype” refers to the cell expression of one or more proteins (e.g., cellular proteomics). In various embodiments, a cell phenotype is determined using a single-cell analysis. In various embodiments, the cell phenotype can refer to the expression of a panel of proteins (e.g., a panel of proteins involved in cancer processes). In various embodiments, the protein panel includes proteins involved in any of the following hematologic malignancies: acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid disease, myeloproliferative neoplasms, or T-cell lymphoma. In various embodiments, the protein panel includes proteins involved in any of the following solid tumors: breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma. Examples proteins in the panel can include any of HLA-DR, CD10, CD117, CD11b, CD123, CD13, CD138, CD14, CD141, CD15, CD16, CD163, CD19, CD193 (CCR3), CD1c, CD2, CD203c, CD209, CD22, CD25, CD3, CD30, CD303, CD304, CD33, CD34, CD4, CD42b, CD45RA, CD5, CD56, CD62P (P-Selectin), CD64, CD68, CD69, CD38, CD7, CD71, CD83, CD90 (Thy1), Fc epsilon RI alpha, Siglec-8, CD235a, CD298-A, B2M-A, GATA3, CSTB, BCR-ABL (b3a2), MYC, BAD, AKT pS473, CASP3, BCL2, MPO, MKI67, INFG, IL2, CDK1, RPS6 pS244, CD49d, CD45, CD8, CD45RO, mouse IgG1, kappa, mouse IgG2a, kappa, mouse IgG2b, kappa, CD103, CD62L, CD11c, CD44, CD27, CD81, CD319 (SLAMF7), CD269 (BCMA), CD99, CD164, KCNJ3, CXCR4 (CD184), CD109, CD53, CD74, HLA-DR, DP, DQ, HLA-A, B, C, ROR1, Annexin A1, or CD20.

The phrase “cell genotype” refers to the genetic makeup of the cell and can refer to one or more genes and/or the combination of alleles homozygous or heterozygous) of a cell. The phrase cell genotype further encompasses one or more mutations of the cell including polymorphisms, single nucleotide polymorphisms (SNPs), single nucleotide variants (SNVs)), insertions, deletions, knock-ins, knock-outs, insertion or deletion mutation (indel), copy number variations (CNVs), duplications, translocations, and loss of heterozygosity (LOH). In various embodiments, a cell phenotype is determined using a single-cell analysis. In various embodiments, the cell phenotype can refer to the expression of a panel of genes (e.g., a panel of genes involved in cancer processes). In various embodiments, the panel includes genes involved in any of the following hematologic malignancies: acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, or T-cell lymphoma. In various embodiments, the panel includes genes involved in any of the following solid tumors: breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma. For example, for acute lymphoblastic leukemia, the following genes are interrogated: ASXL1, GATA2, KIT, PTPN11, TET2, DNMT3A, IDH1, KRAS, RUNX1, TP53, EZH2, IDH2, NPM1, SF3B1, U2AF1, FLT3, JAK2, NRAS, SRSF2, or WT1.

In some embodiments, the discrete entities as described herein are droplets. The terms “emulsion,” “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g. an aqueous phase fluid (e.g., water). In some embodiments, the second fluid phase will be an immiscible (with respect to the first fluid phase) phase carrier fluid. Thus droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, and a variety of other components. The term emulsion may be used to refer to an emulsion produced in, on, or by a microfluidic device and/or flowed from or applied by a microfluidic device.

The term “antibody” encompasses monoclonal antibodies (including full length monoclonal antibodies), polyclonal antibodies, multispecific antibodies (e.g., bispecific antibodies), and antibody fragments that are antigen-binding, e.g., an antibody or an antigen-binding fragment thereof. “Antibody fragment”, and all grammatical variants thereof, as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Example computer program methods to determine identity and similarity between two sequences include, but are not limited to, the GCG program package (Devereux, J., et al., Nucleic Acids Research 12(1): 387 (1984)), BLAST and BLAST 2.0 algorithms (e.g., BLAST X programs), which are described in Altschul et al, Nuc. Acids Res. 25:3389-3402 (1977), and FASTA (Atschul, S. F. et al., J. Molec. Biol. 215:403-410 (1990)). The BLAST X (e.g., BLASTP, BLASTN) programs are publicly available from NCBI and other sources (BLAST Manual, Altschul, S., et al., NCBINLM NIH Bethesda, Md. 20894: Altschul, S., et al., J. Mol. Biol. 215:403-410 (1990). Other methods include the algorithms of Smith & Waterman, Adv. Appl. Math. 2:482 (1981), and Needleman & Wunsch, J. Mol. Biol. 48:443 (1970), etc.

The term “identical” and their variants, as used herein, when used in reference to two or more sequences, refer to the degree to which the two or more sequences (e.g., nucleotide or polypeptide sequences) are the same. In the context of two or more sequences, the percent identity or homology of the sequences or subsequences thereof indicates the percentage of all monomeric units (e.g., nucleotides or amino acids) that are the same at a given position or region of the sequence (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. Another indication that two nucleic acid sequences are substantially identical is that the two molecules or their complements hybridize to each other under stringent hybridization conditions.

The term “block,” “blocking,” “using a block buffer” and their variants, refer generally to any action or process whereby non-specific binding of antibodies or other reagents to the tissue is prevented. For example, non-specific binding prevents visualization of the antigen-antibody binding of interest. Thus, to mitigate nonspecific binding, a blocking step can be carried out before incubation with an antibody. A blocking buffer may be used in a blocking step. A blocking buffer can be a solution of a different protein, mixture of proteins, or other compound that passively adsorbs to remaining binding surfaces. The blocking buffer may reduce background interference and improve the signal-to-noise ratio. For example, an ideal blocking buffer may bind to potential sites of nonspecific interaction, eliminating background altogether, without altering or obscuring the epitope for antibody binding.

The terms “fixing,” “fixative,” and their related variants, refer generally to any action or process whereby cellular morphology, integrity, and/or structure are reserved so as to prevent an autolysis of cells and the process of putrefaction (cellular decay). A fixative may be used to enhance the rigidity and mechanical strength of cells, to withstanding the immunostaining procedure, as described herein. In some embodiments, cells may be fixed immediately following removal from cell culture conditions to limit autolysis and putrefaction.

The terms “permeabilize,” “permeabilizing,” “permeabilization,” “using a permeabilization buffer” and their variants, refer generally to any action or process whereby the cell membrane is punctured where membrane lipids are partially removed or dissolved to allow for at least a portion of the antibodies or any desired molecules to pass through a cellular membrane and enter the cell. A permeabilization buffer may be used in a permeabilizing step. In some embodiments, a permeabilization buffer can be a solution of non-ionic detergent, or other permeabilizing agents, as described herein.

The terms “amplify,” “amplifying,” “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes a sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further include any of the amplification processes known to one of ordinary skill in the art. In some embodiments, the amplification reaction includes polymerase chain reaction (PCR). In some embodiments, the amplification reaction includes an isothermal amplification reaction such as LAMP. In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

Any nucleic acid amplification method may be utilized, such as a PCR-based assay, e.g., quantitative PCR (qPCR), or an isothermal amplification may be used to detect the presence of certain nucleic acids, e.g., genes of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reagent. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers.”

A “barcode” nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

The terms “nucleic acid,” “polynucleotides,” and “oligonucleotides” refers to biopolymers of nucleotides and, unless the context indicates otherwise, includes modified and unmodified nucleotides, and DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes deoxythymidine, and ‘U’ denotes uridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label.” In some embodiments, the label can be in the form of a fluorescent moiety (e.g., dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof.

It must be noted that, as used in the specification, the singular forms “a,” “an” and “the” include plural referents unless the context clearly dictates otherwise.

All references, issued patents and patent applications cited within the body of the specification are hereby incorporated by reference in their entirety, for all purposes.

Overview

Described herein are embodiments for analyzing one or more analytes, such as one or more protein analytes, of an individual cell. In various embodiments, the analyte of the cell can be located on a surface of the cell. In various embodiments, the analyte of the cell can be located internally within the cell. In various embodiments, a first analyte of the cell can be located on a surface of the cell and a second analyte can be located internally within the cell. In various embodiments, the method described herein can be applied to one or more cell lines.

Generally, analyzing the one or more analytes of the cell involves preparing (e.g., washing, staining, fixing, blocking, and/or permeabilizing) the cell, and providing one or more antibody-oligonucleotide conjugates to the prepared cell. Thus, the antibody-oligonucleotide conjugates can be bound to the analyte located on the surface of the cell to generate a surface antibody-oligonucleotide conjugate, and/or enter the permeabilized cell to contact the analyte located internally within the cell to generate an intracellular antibody-oligonucleotide conjugate. Moreover, analyzing the one or more analytes of the cell further involves performing a single-cell analysis of the incubated cell, as described herein.

The single-cell analysis involves generating amplicons derived from the one or more analytes and sequencing the amplicons to determine presence or absence of the analytes. In particular embodiments, the one or more analytes comprise genomic DNA, protein located on the surface of the cell, and/or protein located internally within the cell. The single-cell analysis further involves determining presence or absence of the cell genotype (e.g., cell mutations such as CNVs, indels, and/or SNVs). In various embodiments, to analyze proteins (e.g., surface proteins and/or intracellular proteins), the single cell analysis involves sequencing oligonucleotides that are linked to antibodies, where the antibodies exhibit binding affinity for a specific protein expressed by a cell. Thus, sequence reads derived from the antibody-conjugated oligonucleotides are used to determine the cell phenotype (e.g., expression or presence of one or more analytes of the cell). The single-cell analysis in the present disclosure (e.g., inclusion of intracellular protein detection) can enable measurement of proteins in cancer mechanisms, such as apoptosis (BCL2 family proteins), transcription factors (GATA3), tumor suppressors (TP53), and/or phosphorylated proteins involved in cell growth signaling pathways (e.g., phosphorylated ERK and/or STAT proteins).

Advantageously, in various embodiments, the single-cell analysis in the present disclosure can provide a solution to link surface and intracellular protein measurement with targeted DNA analysis. Thus, single-cell readout of genotypic and phenotypic information can be collected together to enable concurrent complex analyses of cancer clonal evolution and driver protein expression.

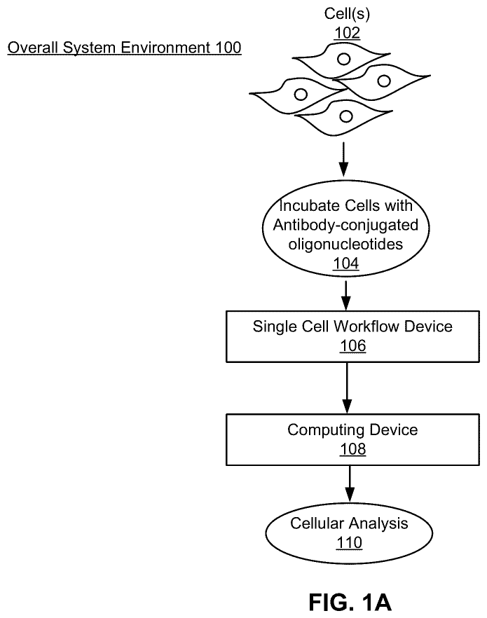

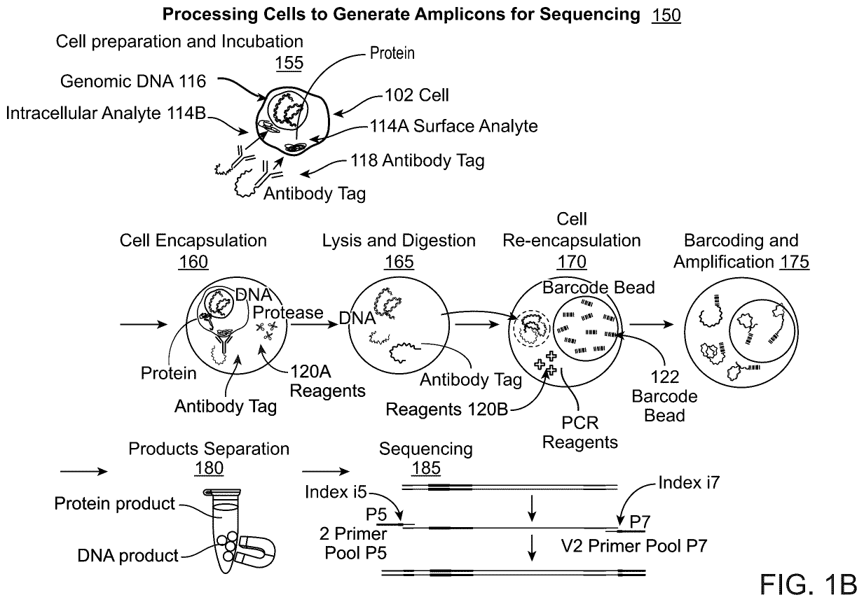

In various embodiments, the FIGS. 1-4 can include additional or fewer components and/or steps. For example, the step 104 in FIG. 1A and/or the step 155 in FIG. 1B need not include incubation for enabling antibodies to bind to surface analytes. In another example, the step 104 in FIG. 1A and/or the step 155 in FIG. 1B need not include incubation for enabling antibodies to bind to intracellular analytes. In another example, the single cell analysis as described herein and in FIGS. 1-4 need not include processing or analyzing surface analytes. In another example, the single cell analysis as described herein and in FIGS. 1-4 need not include processing or analyzing intracellular analytes.

Reference is made to FIG. 1A, which depicts an overall system environment 100 including a single cell workflow device 106 and a computational device 108 for analyzing one or more analytes of one or more individual cells 102, in accordance with an embodiment. In various embodiments, the cells 102 can be isolated from a test sample obtained from a subject or a patient. In various embodiments, the cells 102 are healthy cells taken from a healthy subject. In various embodiments, the cells 102 include diseased cells taken from a subject. In one embodiment, the cells 102 include cancer cells taken from a subject previously diagnosed with cancer. For example, cancer cells can be tumor cells available in the bloodstream of the subject diagnosed with cancer. As another example, cancer cells can be cells obtained through a tumor biopsy. Thus, single-cell analysis of the tumor cells enables analysis of cells of the subject's cancer. In various embodiments, the test sample is obtained from a subject following treatment of the subject (e.g., following a therapy such as cancer therapy). Thus, single-cell analysis of the cells enables analysis of cells representing the subject's response to a therapy. In various embodiments, the cells 102 are or include one or more complete cells. In various embodiments, the cells 102 are or include one or more nuclei and/or partial cells, where the nuclei and/or partial cells are isolated from tissues and/or a suspension of complete cells before the single cell analysis workflow. Example methodologies for isolating cellular nuclei from cells are further described in Nabbi et al., “Isolation of Nuclei.” Cold Spring Harb Protoc. 2015(8): 731-734, and Vindelov et al., “N. I. A detergent-trypsin method for the preparation of nuclei for flow cytometric DNA analysis.” Cytometry 1983(3), 323-327, which are hereby incorporated by reference in its entirety.

At step 104, the cells 102 are prepared, and/or incubated with one or more antibodies. In various embodiments, the antibody is conjugated to the oligonucleotide. In various embodiments, the antibody exhibits binding affinity to a target analyte. For example, the antibody can exhibit binding affinity to a target epitope of a target protein.

In particular embodiments, step 104 involves performing any of washing the cell 102, blocking the cell 102, fixing the cell 102, quenching the cell 102, and/or permeabilizing the cell 102, as described in further detail below.

In various embodiments, washing the cell 102 comprises washing the cell 102 with wash buffer. In various embodiment, washing the cell 102 comprises washing the cell 102 for one or more times. In various embodiment, washing the cell 102 comprises washing the cell 102 for at least 2, 3, 4, 5, 6, 7, 8, 9, or 10 times. In various embodiments washing the cell 102 comprises washing the cell 102 for at least 1 minute, 2 minutes, 3 minutes, 4 minutes, 5 minutes, 6 minutes, 7 minutes, 8 minutes, 9 minutes, or 10 minutes.

In various embodiments, fixing the cell 102 comprises fixing the cell 102 using fixatives for at least 30, 45, 60, or 90 minutes. In particular embodiments, fixing the cell 102 comprises fixing the cell 102 using fixatives for 90 minutes. In various embodiments, fixing the cell 102 comprises fixing the cell 102 at a temperature between 4 and 50° C. In various embodiments, fixing the cell 102 comprises fixing the cell 102 at a temperature between 10 and 30° C. In various embodiments, fixing the cell 102 comprises fixing the cell 102 at a temperature between 20 and 25° C. In various embodiments, fixing the cell 102 comprises fixing the cell 102 at a temperature between 20 and 25° C. for 90 minutes. In various embodiments, fixing the cell 102 comprises fixing the cell 102 using 0.1 mM to 20 mM of one or more fixatives in a reactive volume using a background buffer. In various embodiments, fixing the cell 102 comprises fixing the cell 102 using 0.5 mM to 10 mM of one or more fixatives in a reactive volume using a background buffer. In various embodiments, fixing the cell 102 comprises fixing the cell 102 using 1 mM to 5 mM of one or more fixatives in a reactive volume using a background buffer. In various embodiments, the reactive volume is from 0.01 to 10 mL. In various embodiments, the reactive volume is from 0.05 to 5 mL. In particular embodiments, the reactive volume is from 0.1 to 1 mL. In particular embodiments, the background buffer is Dulbecco's phosphate-buffered saline (DPBS).

In various embodiments, quenching the cell 102 comprises quenching the fixed cell for at least 1, 2, 3, 4, 5, 6, 7, 8, 9, or 10 minutes. In various embodiments, quenching the cell 102 comprises quenching the fixed cell at a temperature between 10 and 50° C. In various embodiments, quenching the cell 102 comprises quenching the fixed cell at a temperature between 10 and 30° C. In various embodiments, quenching the cell 102 comprises quenching the fixed cell at a temperature between 20 and 25° C.

In various embodiments, blocking the cell 102 comprises blocking the cell 102 for at least 10, 20, or 30 minutes. In various embodiments, blocking the cell 102 comprises blocking the cell 102 at a temperature between 10 and 50° C. In various embodiments, blocking the cell 102 comprises blocking the cell 102 at a temperature between 10 and 30° C. In various embodiments, blocking the cell 102 comprises blocking the cell 102 at a temperature between 20 and 25° C. In various embodiments, blocking the cell 102 comprises using a blocking buffer. In particular embodiments, the blocking buffer is used in the surface protein product for preparing the cell.

In various embodiments, permeabilizing the cell 102 comprises permeabilizing the cell 102 for at least 10, 20, or 30 minutes. In various embodiments, permeabilizing the cell 102 comprises permeabilizing the cell 102 at a temperature between 10 and 50° C. In various embodiments, permeabilizing the cell 102 comprises permeabilizing the cell 102 at a temperature between 10 and 30° C. In various embodiments, permeabilizing the cell 102 comprises permeabilizing the cell 102 at a temperature between 20 and 25° C. In various embodiments, permeabilizing the cell 102 comprises permeabilizing the cell 102 using a permeabilization buffer. In various embodiments, the permeabilization buffer comprises a 0.01%, 0.05%, 0.1%, 0.15%, or 0.2% solution. In various embodiments, the permeabilization buffer comprises at least one of Triton™ X-100, Prionex® gelatin, salmon sperm DNA, mouse IgG, EDTA. In various embodiments, the permeabilization buffer comprises Triton™ X-100. In particular embodiments, the permeabilization buffer comprises 0.1% Triton™ X-100.

In various embodiments, incubating the cell 102 with antibodies include incubating the cell 102 with antibody-conjugated oligonucleotides. In various embodiments, the antibody-conjugated oligonucleotide binds to the analyte located on the surface of the cell to generate a surface antibody-oligonucleotide conjugate. In various embodiments, the antibody-oligonucleotide conjugate enters the permeabilized cell to contact the analyte located internally within the cell to generate an intracellular antibody-oligonucleotide conjugate. In various embodiments, the antibody-conjugated oligonucleotide binds to the analyte located on the surface of the cell to generate a surface antibody-oligonucleotide conjugate, and enters the permeabilized cell to contact the analyte located internally within the cell to generate an intracellular antibody-oligonucleotide conjugate.

In various embodiments, incubating the cell 102 with antibodies includes incubating the cell 102 with antibody-oligonucleotide conjugates (e.g., antibody tag 118 in FIG. 1B) for 10 minutes to 30 hours. In various embodiments, incubating the cell 102 with antibodies includes incubating the cell 102 with antibody-oligonucleotide conjugates (e.g., antibody tag 118 in FIG. 1B) for 10-60 minutes. In various embodiments, incubating the cell 102 with antibodies includes incubating the cell 102 with antibody-oligonucleotide conjugates (e.g., antibody tag 118 in FIG. 1B) for 30 minutes. In various embodiments, incubating the cell 102 with antibodies includes incubating the cell 102 with antibody-oligonucleotide conjugates (e.g., antibody tag 118 in FIG. 1B) for 10-25 hours. In various embodiments, incubating the cell 102 with antibodies includes incubating the cell 102 with antibody-oligonucleotide conjugates (e.g., antibody tag 118 in FIG. 1B) for 16-20 hours. In various embodiments, incubating the cell 102 with antibodies includes incubating the cell 102 with antibody-oligonucleotide conjugates (e.g., antibody tag 118 in FIG. 1B) overnight.

In various embodiments, incubating the cell 102 with antibodies includes incubating the cell 102 with antibody-oligonucleotide conjugates (e.g., antibody tag 118 in FIG. 1B) at a temperature between 0-30° C. In various embodiments, incubating the cell 102 with antibodies includes incubating the cell 102 with antibody-oligonucleotide conjugates at a temperature between 2-30° C. In various embodiments, incubating the cell 102 with antibodies includes incubating the cell 102 with antibody-oligonucleotide conjugates at a temperature between 3-6° C. In various embodiments, incubating the cell 102 with antibodies includes incubating the cell 102 with antibody-oligonucleotide conjugates at a temperature of about 4° C. In particular embodiments, incubating the cell 102 with antibodies includes incubating the cell 102 with antibody-oligonucleotide conjugates at room temperature (e.g., about 22° C.). In particular embodiments, incubating the cell 102 with antibodies includes incubating the cell 102 with antibody-oligonucleotide conjugates on ice (e.g., at about 0° C.).

In various embodiments, the number of cells incubated with antibodies can be 10² cells, 10³ cells, 10⁴ cells, 10⁵ cells, 10⁶ cells, or 10⁷ cells. In various embodiments, between 10³ cells and 10⁷ cells are incubated with antibodies. In various embodiments, between 10⁴ cells and 10⁶ cells are incubated with antibodies. In various embodiments, varying concentrations of antibodies are incubated with cells. In various embodiments, for an antibody in the protein panel, a concentration of 0.1 nM, 0.5 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, or 100 nM of the antibody is incubated with cells.

In various embodiments, cells 102 are incubated with a plurality of different antibodies. In one embodiment, amongst the plurality of different antibodies, each antibody exhibits binding affinity for an analyte of a panel. For example, each antibody exhibits binding affinity for a protein of a panel. Examples of proteins included in protein panels are described herein. The incubation of cells with antibodies leads to the binding of the antibodies against target epitopes. In various embodiments, a concentration of 0.05 nM, 0.1 nM, 0.5 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, or 100 nM for each antibody of the antibody panel is incubated with cells.

Following incubation, the cells 102 may be washed (e.g., with a wash buffer) for one or more times to remove excess antibodies that are unbound. In various embodiments, the cells 102 are washed for at least 1 minute, 2 minutes, 3 minutes, 4 minutes, 5 minutes, 6 minutes, 7 minutes, 8 minutes, 9 minutes, 10 minutes, 15 minutes, or 20 minutes to wash away unbound antibody-oligonucleotide conjugates. In various embodiments, the cells 102 are washed for at least 2, 3, 4, 5, 6, 7, 8, 9 or 10 times to wash away unbound antibody-oligonucleotide conjugates. In particular embodiments, the cells 102 are washed for 4 times. In particular embodiments, the cells 102 are washed for 5 minutes. In particular embodiments, the cells 102 are washed for 5 minutes for 4 times.

In various embodiments, the antibodies are labeled with one or more oligonucleotides, also referred to as antibody oligonucleotides. Such oligonucleotides can be read out with microfluidic barcoding and DNA sequencing, thereby enabling the detection of cell analytes of interest. When an antibody binds its target, the antibody oligonucleotide is carried with it and thus allows the presence of the target analyte to be inferred based on the presence of the oligonucleotide tag. In some implementations, analyzing antibody oligonucleotides provides an estimate of the different epitopes present in the cell.

The single cell workflow device 106 refers to a device that processes individuals cells to generate amplicons for sequencing. In various embodiments, the single cell workflow device 106 can encapsulate individual cells into a first droplet, lyse cells within the first droplet, perform cell barcoding of cell lysate in a second droplet, and generate amplicons in the second droplet. Thus, amplicons can be collected and sequenced. In various embodiments, the single cell workflow device 106 further includes a sequencer for sequencing the amplicons. In various embodiments, at least 10, 50, 100, 150, 20, 250, 300, 350, 400, 450, or 500 amplicons (e.g., DNA amplicons and/or amplicons derived from antibody oligonucleotides) are generated in a workflow. In various embodiments, the single cell workflow device 106 can be applied to one or more cell lines. In various embodiments, the single cell workflow device 106 can be applied to at least 2, 3, 4, 5, 6 cell lines, or their combinations thereof. In particular embodiments, the one or more cell lines include HL60, K562, KCL22, Jurkat, T47D, KG1, A549, and/or their mixture and/or mergers thereof.

The computing device 108 is configured to receive the sequenced reads from the single cell workflow device 106. In various embodiments, the computing device 108 is communicatively coupled to the single cell workflow device 106 and therefore, directly receives the sequence reads from the single cell workflow device 106. The computing device 108 analyzes the sequence reads to generate a cellular analysis 110. In one embodiment, the computing device 108 analyzes the sequence reads to determine presence or absence of the analytes. For example, the computing device analyzes the sequence reads to determine presence or absence of surface proteins and/or intracellular proteins. In one embodiment, the computing device 108 analyzes the sequence reads to determine cellular genotypes and phenotypes. The computing device 108 uses the determined cellular genotypes and phenotypes to discover new cell subpopulations and/or to classify individual cells into cell subpopulations. Thus, in such embodiments, the cellular analysis 110 can refer to the identification of cell subpopulations or the classifications of cells into cell subpopulations. In one embodiment, the computing device 108 analyzes the sequence reads to determine one or more mutations such as single-nucleotide polymorphism (SNV), insertion or deletion mutation (indel), or copy number variation (CNV).

Reference is now made to FIG. 1B, which depicts one embodiment of a single cell analysis workflow that includes processing one or more individual cells to generate amplicons for sequencing 150. Specifically, FIG. 1B depicts a workflow process 150 including steps of cell preparation and incubation 155, cell encapsulation 160, lysis and digestion 165, cell re-encapsulation 170, barcoding and amplification 175, products separation 180, and indexing 185, as described herein.

Generally, the step 155 involves incubating one or more individual cells 102 with antibody-conjugated oligonucleotides using one or more antibody tags 118, as noted above in step 104 in FIG. 1A. In various embodiments, the cell 102 comprises a surface analyte (e.g., surface protein) 114A. In various embodiments, the cell 102 comprises an intracellular analyte (e.g., intracellular protein) 114B. In various embodiments, the cell 102 comprises a surface analyte (e.g., surface protein) 114A and an intracellular analyte (e.g., intracellular protein) 114B. In various embodiments, the cell 102 further comprises genomic DNA 116. In various embodiments, the single cell 102 is prepared prior to the step 155 (e.g., preparation in above-noted step 104 in FIG. 1A).

In various embodiments, the step 155 comprises providing one or more first antibody-oligonucleotide conjugates to a cell (e.g., a prepared cell), wherein the one or more first antibody-oligonucleotide conjugates are bound to surface(s) of the cell to generate surface antibody-oligonucleotide conjugates. In particular embodiments, the step 155 comprises providing a first antibody-oligonucleotide conjugate to a cell (e.g., a prepared cell), wherein the first antibody-oligonucleotide conjugate is bound to a surface of the cell to generate a surface antibody-oligonucleotide conjugate. In various embodiments, the step 155 comprises providing one or more additional first antibody-oligonucleotide conjugates specific for one or more additional surface analytes to the prepared cell. In various embodiments, the one or more additional first antibody-oligonucleotide conjugates comprise two additional antibody-oligonucleotide conjugates specific for two surface analytes. In various embodiments, the one or more additional first antibody-oligonucleotide conjugates comprise three additional antibody-oligonucleotide conjugates specific for three surface analytes. In various embodiments, the one or more additional first antibody-oligonucleotide conjugates comprise four additional antibody-oligonucleotide conjugates specific for four surface analytes. In various embodiments, the one or more additional first antibody-oligonucleotide conjugates comprise five additional antibody-oligonucleotide conjugates specific for five surface analytes. In various embodiments, the one or more additional first antibody-oligonucleotide conjugates comprise six additional antibody-oligonucleotide conjugates specific for six surface analytes. In various embodiments, the one or more additional first antibody-oligonucleotide conjugates comprise ten additional antibody-oligonucleotide conjugates specific for ten surface analytes. In various embodiments, the one or more additional first antibody-oligonucleotide conjugates comprise twenty additional antibody-oligonucleotide conjugates specific for twenty surface analytes. In various embodiments, the one or more additional first antibody-oligonucleotide conjugates comprise thirty additional antibody-oligonucleotide conjugates specific for thirty surface analytes. In various embodiments, the one or more additional first antibody-oligonucleotide conjugates comprise forty additional antibody-oligonucleotide conjugates specific for forty surface analytes. In particular embodiments, the one or more additional first antibody-oligonucleotide conjugates comprise forty-five additional antibody-oligonucleotide conjugates specific for forty-five surface analytes. In various embodiments, the one or more additional first antibody-oligonucleotide conjugates comprise fifty additional antibody-oligonucleotide conjugates specific for fifty surface analytes.

In various embodiments, the step 155 comprises providing one or more second antibody-oligonucleotide conjugates to a cell (e.g., a prepared cell, and/or a permeabilized cell), wherein the one or more second antibody-oligonucleotide conjugates enter the cell (e.g., a permeabilized cell) to contact the analyte(s) located internally within the cell to generate intracellular antibody-oligonucleotide conjugates. In particular embodiments, the step 155 comprises permeabilizing a cell (e.g., a prepared cell) and/or providing a second antibody-oligonucleotide conjugate to the permeabilized cell, wherein the second antibody-oligonucleotide conjugate enters the permeabilized cell to contact the analyte located internally within the permeabilized cell to generate an intracellular antibody-oligonucleotide conjugate. In various embodiments, the step 155 comprises providing one or more additional second antibody-oligonucleotide conjugates specific for one or more additional intracellular analytes to the prepared cell. In various embodiments, the one or more additional second antibody-oligonucleotide conjugates comprise five additional antibody-oligonucleotide conjugates specific for five intracellular analytes. In various embodiments, the one or more additional second antibody-oligonucleotide conjugates comprise ten additional antibody-oligonucleotide conjugates specific for ten intracellular analytes. In various embodiments, the one or more additional second antibody-oligonucleotide conjugates comprise fifteen additional antibody-oligonucleotide conjugates specific for fifteen intracellular analytes. In various embodiments, the one or more additional second antibody-oligonucleotide conjugates comprise twenty additional antibody-oligonucleotide conjugates specific for twenty intracellular analytes. In various embodiments, the one or more additional second antibody-oligonucleotide conjugates comprise thirty additional antibody-oligonucleotide conjugates specific for thirty intracellular analytes. In various embodiments, the one or more additional second antibody-oligonucleotide conjugates comprise forty additional antibody-oligonucleotide conjugates specific for forty intracellular analytes. In particular embodiments, the one or more additional second antibody-oligonucleotide conjugates comprise forty-five additional antibody-oligonucleotide conjugates specific for forty-five intracellular analytes. In various embodiments, the one or more additional second antibody-oligonucleotide conjugates comprise fifty additional antibody-oligonucleotide conjugates specific for fifty intracellular analytes. In particular embodiments, the one or more second antibody-oligonucleotide conjugates comprise a concentration of up to 13 nM per antibody.

Generally, the step 160 involves encapsulating the incubated cell with first reagents 120A into a first droplet. The step 165 involves lysing and digesting the encapsulated cell to release an analyte in the encapsulated cell within the first droplet. In various embodiments, the encapsulated cell is a prepared, incubated, and/or permeabilized cell within the first droplet. In particular embodiments, lysing the permeabilized cell within the first droplet comprises optionally applying extra reagents. In various embodiments, the extra reagents comprise dithiothreitol (DTT). In various embodiments, the amount of the extra reagents is less than 0.5, 1, 2, 3, 4, 5, 6, 7, 8, 9, or 10 mM DTT. In particular embodiments, the amount of the extra reagents is less than 5 mM DTT.

As shown in FIG. 1B, the first reagents 120A cause the encapsulated cell to lyse, thereby generating a cell lysate within the first droplet. In various embodiments, the cell lysate comprises surface protein, intracellular protein, and/or genomic DNA. In particular embodiments, the step 165 comprises lysing the encapsulated cell with the first droplet to generate a cell lysate comprising a first oligonucleotide or a complement of the first oligonucleotide from the first antibody-oligonucleotide conjugate generated from the step 155. In particular embodiments, the step 165 comprises lysing the encapsulated cell with the first droplet to generate a cell lysate comprising a second oligonucleotide or a complement of the second oligonucleotide from the second antibody-oligonucleotide conjugate generated from the step 155. In particular embodiments, the step 165 comprises lysing the encapsulated cell with the first droplet to generate a cell lysate comprising a first oligonucleotide or a complement of the first oligonucleotide from the first antibody-oligonucleotide conjugate and a second oligonucleotide or a complement of the second oligonucleotide from the second antibody-oligonucleotide conjugate, wherein the first and the second antibody-oligonucleotide conjugates are generated from the step 155. In particular embodiments, the first reagents 120A include proteases, such as proteinase K, for lysing the cell to generate a cell lysate. The cell lysate includes the contents of the cell, which can include one or more different types of analytes (e.g., RNA transcripts, DNA, protein, lipids, or carbohydrates). In various embodiments, the different analytes of the cell lysate can interact with the first reagents 120A within the first droplet. For example, primers in the first reagents 120, such as reverse primers, can prime the analytes.

The step 170 involves encapsulating the cell lysate into a second droplet along with second reagents 120B and/or a barcode bead 122 to generate one or more amplicons. As shown in FIG. 1B, in some embodiments, the second reagents 120B and the barcode bead 122 can be separately introduced in individual regions within the second droplet. In various embodiments, the barcode bead 122 comprises one or more barcodes, as described herein. Generally, a barcode can label a target analyte to be analyzed, which enables subsequent identification of the origin of a sequence read that is derived from the target analyte. In various embodiments, multiple barcodes can label multiple target analytes of the cell lysate, thereby enabling the subsequent identification of the origin of large quantities of sequence reads. In particular embodiments, the second reagents 120B include reagents for performing a reaction, such as a nucleic acid amplification reaction. For example, the second reagents 120B can include dNTPs and/or primers.

Generally, the step 175 involves generating one or more amplicons from the one or more oligonucleotides or the complement of the one or more oligonucleotides by performing at least one reaction, such as a nucleic acid amplification reaction, using the reagents. In various embodiments, the step 175 further involves generating one or more amplicons from genomic DNA. Although FIG. 1B depicts cell barcoding 170 and target amplification 175 as two separate steps, in various embodiments, the target analyte can be labeled with a barcode through the amplification step. In various embodiments, the one or more amplicons are generated from the first oligonucleotide or the complement of the first oligonucleotide (e.g., from a surface antibody-oligonucleotide conjugate) generated from step 165 by performing a reaction using the second reagents 120B. In various embodiments, the one or more amplicons are generated from the second oligonucleotide or the complement of the second oligonucleotide (e.g., from an intracellular antibody-oligonucleotide conjugate) generated from step 165 by performing a reaction using the second reagents 120B. In various embodiments, the one or more amplicons are generated from the first and the second oligonucleotides or the complement of the first and the second oligonucleotides generated from step 165 by performing a reaction using the second reagents 120B. In various embodiments, the at least one reaction comprises nucleic acid amplification, polymerase chain reaction (PCR), and/or loop-mediated isothermal amplification (LAMP).

Generally, the step 180 involves separating (e.g., by size) multiple omics or analyte products in the target analytes for sequencing. In various embodiments, the step 180 is optional or can be absent.

Generally, the step 185 involves sequencing the one or more amplicons generated from the step 170 to determine presence or absence of the analyte of the cell, and/or to determine presence or absence of one or more mutations (e.g., SNV, indels, CNV, etc) based on the sequenced amplicons generated from the genomic DNA. In some embodiments, the step 185 involves measuring one or more proteins in cancer mechanisms, such as apoptosis (BCL2 family proteins), transcription factors (GATA3), tumor suppressors (TP53), and/or phosphorylated proteins involved in cell growth signaling pathways (e.g., phosphorylated ERK and/or STAT proteins).

As referred herein, the workflow process shown in FIG. 1B is a two-step workflow process in which the step 165 (e.g., lysing and digesting) occurs separate from the steps 170 (e.g., adding barcodes and reagents) and 175 (e.g. target amplification). For example, step 165 (e.g., lysing and digesting) occurs within a first droplet followed by the steps 170 (e.g., adding barcodes and reagents) and 175 (e.g. target amplification) in a second droplet. In various embodiments, alternative workflow processes (e.g., workflow processes other than the two-step workflow process shown in FIG. 1B) can be employed. For example, the cell 102, reagents 120A and 120B, and barcode bead 122 can be encapsulated in a droplet. Thus, step 165 (e.g., lysing and digesting) can occur within the droplet, followed by cell the steps 170 (e.g., adding barcodes and reagents) and 175 (e.g. target amplification) within the same droplet.

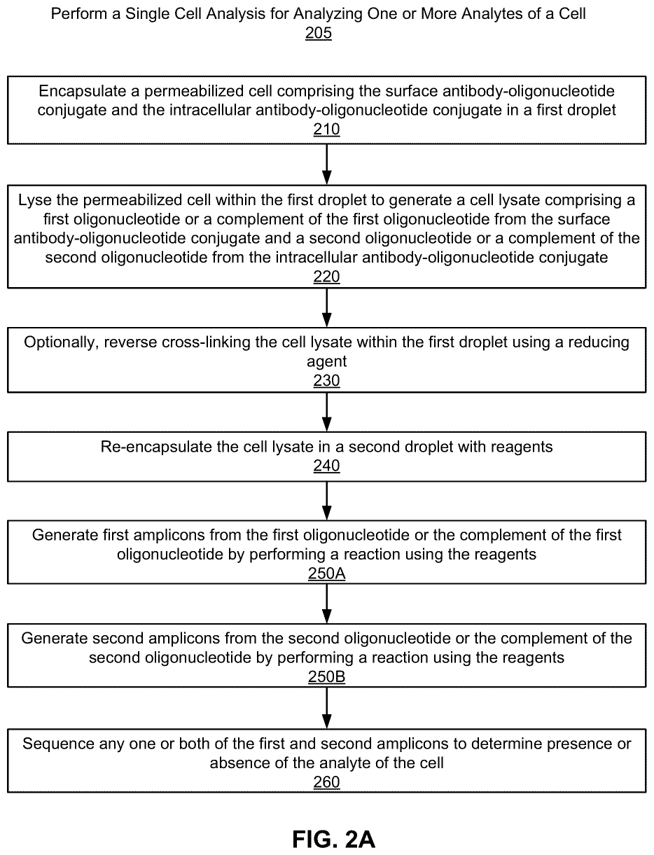

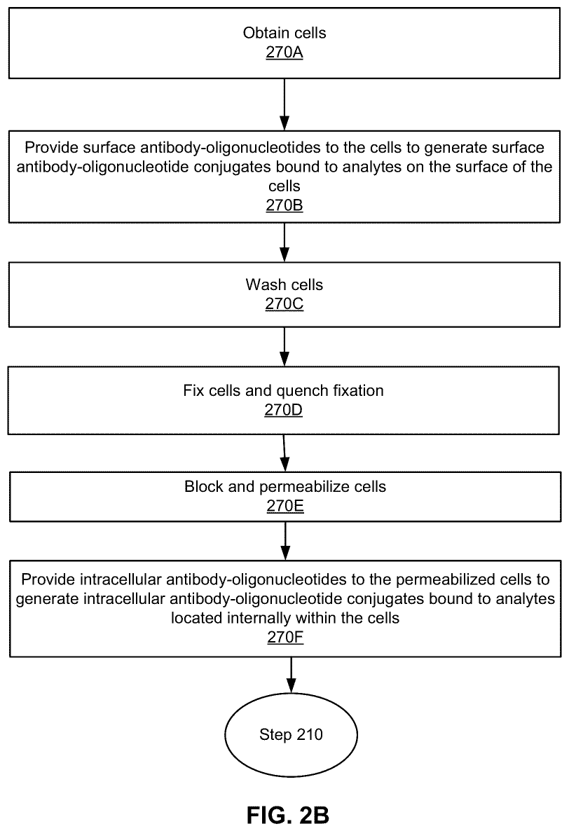

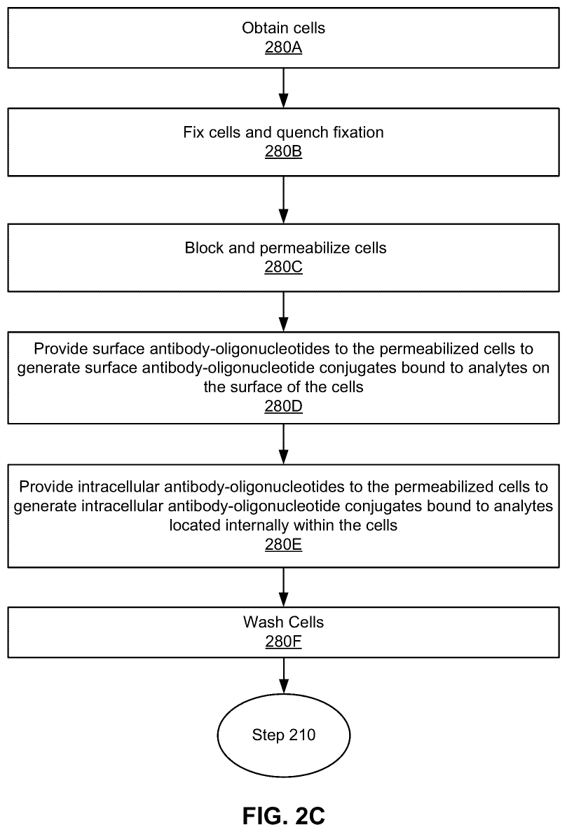

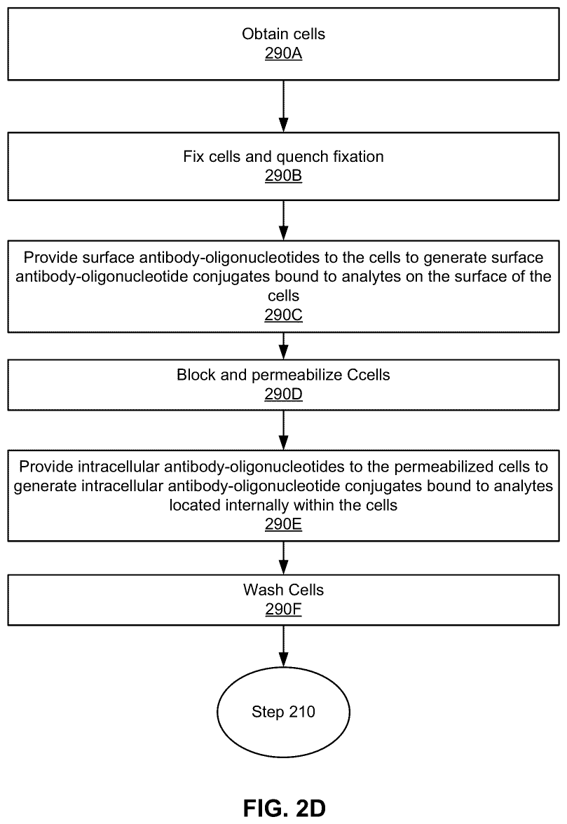

FIG. 2A is a flow process for performing a single cell analysis for analyzing one or more analytes of a cell. Generally, the flow process shown in FIG. 2A elaborates upon steps 160, 165, 170, 175, and 185 in FIG. 1B in further detail, as described herein. Therefore, in various embodiments, the cell has been prepared (e.g., washed, blocked, stained, fixed, and/or permeabilized) and incubated prior to the single cell analysis.

In particular embodiments, the single cell analysis is performed for a least 1 million cells in one workflow.

At step 210, a permeabilized cell comprising the surface antibody-oligonucleotide conjugate and the intracellular antibody-oligonucleotide conjugate is encapsulated in a first droplet. As noted above, in various embodiments, the permeabilized cell need not comprise surface antibody-oligonucleotide conjugate. In various embodiments, the permeabilized cell need not include intracellular antibody-oligonucleotide conjugate.

At step 220, the permeabilized cell is lysed within the first droplet to generate a cell lysate comprising a first oligonucleotide or a complement of the first oligonucleotide from the first antibody-oligonucleotide conjugate and a second oligonucleotide or a complement of the second oligonucleotide from the second antibody-oligonucleotide conjugate. As noted above, in various embodiments, the cell lysate need not comprise the first oligonucleotide or a complement of the first oligonucleotide from the first antibody-oligonucleotide conjugate. In various embodiments, the cell lysate need not comprise the second oligonucleotide or a complement of the first oligonucleotide from the second antibody-oligonucleotide conjugate.

At step 230, the cell lysate is optionally reverse cross-linked within the first droplet using a reducing agent.

At step 240, the cell lysate is re-encapsulated in a second droplet with reagents.

At step 250A, first amplicons are generated from the first oligonucleotide or the complement of the first oligonucleotide by performing a reaction using the reagents.

At step 250B, second amplicons are generated from the second oligonucleotide or the complement of the second oligonucleotide by performing a reaction using the reagents.

At step 260, any one or both of the first and second amplicons are sequenced to determine presence or absence of the analyte of the cell.

### Example Methods for Preparing and Incubating Cells

Reference is now made to FIGS. 2B-2D which show flow processes for preparing and incubating cells (e.g., with antibody oligonucleotides). FIGS. 2B-2D are generally performed prior to the flow process shown in FIG. 2A.

FIG. 2B shows a flow a process of performing a single cell analysis for analyzing one or more analytes of a cell, in accordance with a first embodiment. Here, FIG. 2B shows one embodiment in preparing cells that are then encapsulated (e.g., at step 210 as described in FIG. 2A). In this embodiment, surface antibody-oligonucleotide conjugates and intracellular antibody-oligonucleotide conjugates are separately provided to the cells. Specifically, at step 270A, cells are obtained e.g., in bulk. In some embodiments, step 270A involves blocking the cells. Step 270B involves providing surface antibody-oligonucleotide conjugates to the cells to generate surface antibody-oligonucleotide conjugates bound to analytes on the surface of the cells. Step 270C involves washing the cells to remove unbound surface antibody-oligonucleotide conjugates. Step 270D involves fixing cells and/or quenching fixation. In some embodiments. Step 270E involves blocking and permeabilizing the cells. Step 270F involves providing intracellular antibody-oligonucleotides to the permeabilized cells to generate intracellular antibody-oligonucleotide conjugates bound to analytes located internally within the cells. Optionally, after step 270F, the cells are further washed to remove unbound intracellular antibody-oligonucleotide conjugates. Thus, the permeabilized cell comprising the surface antibody-oligonucleotide conjugate and the intracellular antibody-oligonucleotide conjugate can be encapsulated at step 210.

FIG. 2C shows a flow process of performing a single cell analysis for analyzing one or more analytes of a cell, in accordance with a second embodiment. Here, FIG. 2C shows one embodiment in preparing cells that are then encapsulated (e.g., at step 210 as described in FIG. 2A). In this embodiment, surface antibody-oligonucleotide conjugates and intracellular antibody-oligonucleotide conjugates are provided to the cells together (or provided sequentially without any steps in between them). Specifically, at step 280A, cells are obtained e.g., in bulk. Step 280B involves fixing the cells and/or quenching the fixation. In some embodiments, step 280B is included in step 280A. Step 280C involves blocking and permeabilizing the cells. Step 280D involves providing surface antibody-oligonucleotide conjugates to the cells to generate surface antibody-oligonucleotide conjugates bound to analytes on the surface of the cells. Step 280E involves providing intracellular antibody-oligonucleotides to the permeabilized cells to generate intracellular antibody-oligonucleotide conjugates bound to analytes located internally within the cells. In various embodiments, the order of steps 280D and 280E are reversed. In various embodiments, steps 280D and 280E are a single step performed simultaneously. Step 280F involves washing the cells to remove unbound surface antibody-oligonucleotide conjugates and unbound intracellular antibody-oligonucleotide conjugates. Thus, the permeabilized cell comprising the surface antibody-oligonucleotide conjugate and the intracellular antibody-oligonucleotide conjugate can be encapsulated at step 210.

FIG. 2D shows a flow a process of performing a single cell analysis for analyzing one or more analytes of a cell, in accordance with a first embodiment. Here, FIG. 2D shows one embodiment in preparing cells that are then encapsulated (e.g., at step 210 as described in FIG. 2A). In this embodiment, surface antibody-oligonucleotide conjugates and intracellular antibody-oligonucleotide conjugates are separately provided to the cells. Specifically, at step 290A, cells are obtained e.g., in bulk. Step 290B involves fixing cells and/or quenching fixation. In some embodiments, step 290B is included in step 290A. In some embodiments. Step 290C involves providing surface antibody-oligonucleotide conjugates to the cells to generate surface antibody-oligonucleotide conjugates bound to analytes on the surface of the cells. Step 290D involves blocking and permeabilizing the cells. Step 290E involves providing intracellular antibody-oligonucleotides to the permeabilized cells to generate intracellular antibody-oligonucleotide conjugates bound to analytes located internally within the cells. Step 290F involves washing the cells to remove unbound surface antibody-oligonucleotide conjugates and/or unbound intracellular antibody-oligonucleotide conjugates. Thus, the permeabilized cell comprising the surface antibody-oligonucleotide conjugate and the intracellular antibody-oligonucleotide conjugate can be encapsulated at step 210.

Methods for Performing Single-Cell Analysis

### Encapuslation, Analyte Release, Barcoding, and Amplification

Embodiments described herein involve encapsulating one or more cells (e.g., at step 160 in FIG. 1B) to perform single-cell analysis on the one or more cells. In various embodiments, encapsulating a cell with reagents is accomplished by combining an aqueous phase including the cell and reagents with an immiscible oil phase. In one embodiment, an aqueous phase including the cell and reagents are flowed together with a flowing immiscible oil phase such that water in oil emulsions are formed, where at least one emulsion includes a single cell and the reagents. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, the aqueous phase including the cell and reagents need not be simultaneously flowing with the immiscible oil phase. For example, the aqueous phase can be flowed to contact a stationary reservoir of the immiscible oil phase, thereby enabling the budding of water in oil emulsions within the stationary oil reservoir.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell and reagents within an emulsion can then be flowed through the microfluidic device to undergo cell lysis.

Further example embodiments of adding reagents and cells to emulsions can include merging emulsions that separately contain the cells and reagents or picoinjecting reagents into an emulsion. Further description of example embodiments is described in U.S. application Ser. No. 14/420,646, which is hereby incorporated by reference in its entirety.

The encapsulated cell in an emulsion is lysed to generate cell lysate. In various embodiments, a cell is lysed by lysing agents that are present in the reagents. For example, the reagents can include a detergent such as NP-40 and/or a protease. The detergent and/or the protease can lyse the cell membrane. In some embodiments, cell lysis may also, or instead, rely on techniques that do not involve a lysing agent in the reagent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient means of effecting cell lysis may be employed in the methods described herein.

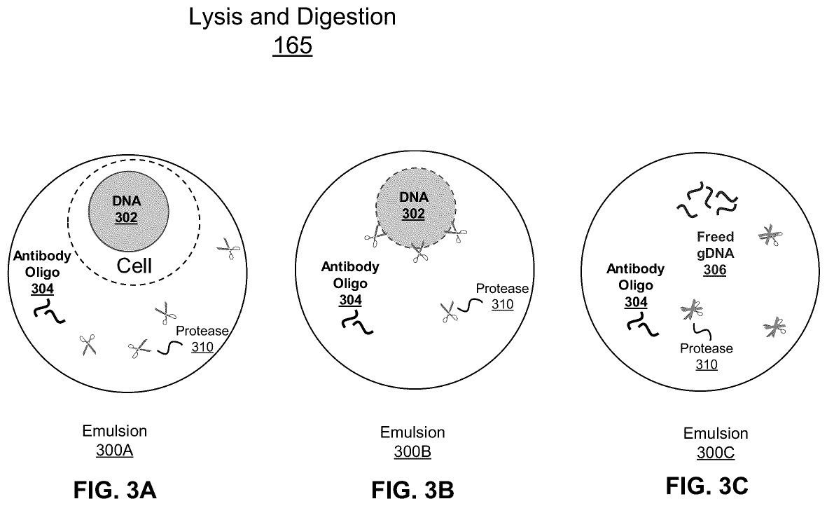

Reference is now made to FIGS. 3A-3C, which depict steps of releasing and processing analytes within an emulsion or a droplet (e.g., emulsion 300), in accordance with a first embodiment. FIG. 3A depicts emulsion 300A that includes both the cell 102 and reagents 120 (as shown in FIG. 1B). Specifically, in FIG. 3A, the emulsion 300A contains the cell (which further includes DNA 302), antibody oligonucleotides 304 (from the antibodies used to bind cell proteins at step 104 in FIG. 1A), as well as proteases 310 that are added from the reagents. Within the emulsion 300A, the cell is lysed, as indicated by the dotted line of the cell membrane. In one embodiment, the cell is lysed by detergents included in the reagents, such as NP40 (e.g., 0.01% NP40).

FIG. 3B depicts the emulsion 300B as the proteases 310 digest the chromatin-bound DNA 302, thereby releasing genomic DNA. In various embodiments, emulsion 300B is exposed to elevated temperatures to enable the proteases 310 to digest the chromatin. In various embodiments, emulsion 300B is exposed to a temperature between 40° C. and 60° C. In various embodiments, emulsion 300B is exposed to a temperature between 45° C. and 55° C. In various embodiments, emulsion 300B is exposed to a temperature between 48° C. and 52° C. In various embodiments, emulsion 300B is exposed to a temperature of 50° C.

FIG. 3C depicts the free genomic DNA strands 306 and the antibody oligonucleotides 304 residing within emulsion 300C. Proteases 310 are inactivated. In various embodiments, proteases 310 are inactivated by exposing emulsion 300C to an elevated temperature. In various embodiments, emulsion 300C is exposed to a temperature between 70° C. and 90° C. In various embodiments, emulsion 300C is exposed to a temperature between 75° C. and 85° C. In various embodiments, emulsion 300C is exposed to a temperature between 78° C. and 82° C. In various embodiments, emulsion 300C is exposed to a temperature of 80° C.

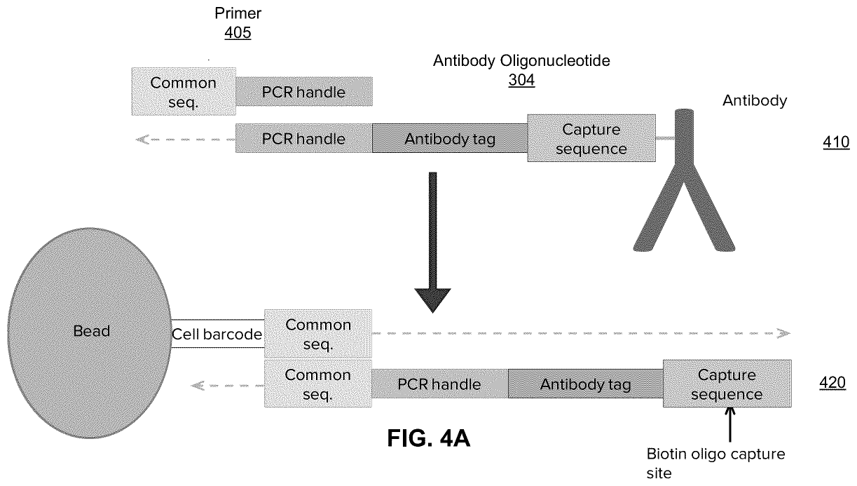

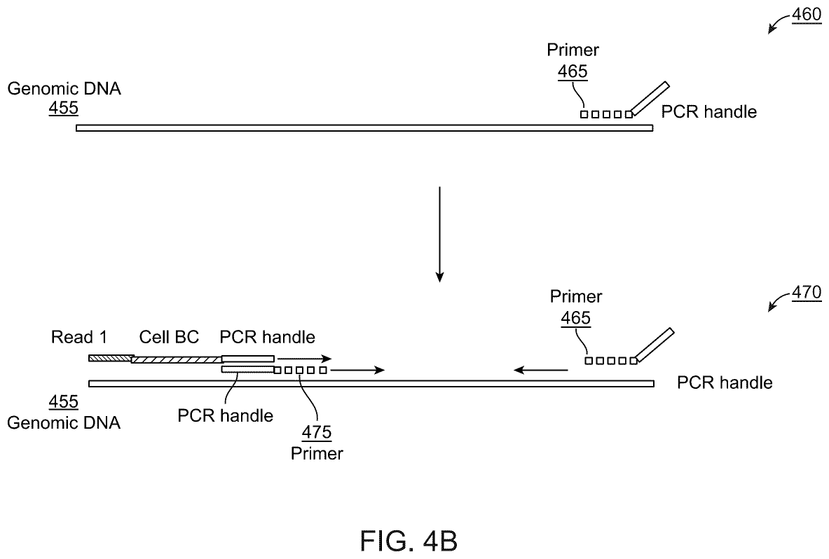

In various embodiments, the antibody oligonucleotide 304 and/or the free genomic DNA 306 undergo priming within emulsion 300C. In various embodiments, reverse primers can hybridize with a portion of the antibody oligonucleotide 304 and/or the free genomic DNA 306. For example, the reverse primer is a gene specific reverse primer that hybridizes with a portion of the free genomic DNA 306. Examples gene specific primers are described in further detail below. As another example, the reverse primer is a PCR handle that hybridizes with a portion of the antibody oligonucleotide 304, which is described in further detail below in relation to FIG. 4A. In various embodiments, the priming of the antibody oligonucleotide 304 can occur earlier, for example in emulsion 300A or emulsion 300B, given that the reverse primers are included in the reagents, which are introduced into emulsion 300A along with the proteases 310.

In various embodiments, the antibody oligonucleotide 304 and the free genomic DNA 306 in emulsion 300C represent at least in part the cell lysate, such as cell lysate shown in FIG. 1B, which is subsequently encapsulated in a second emulsion for barcoding and amplification.

Once the reagents and barcode are added to an emulsion, the emulsion may be incubated under conditions that facilitate the nucleic acid amplification reaction. In various embodiments, the emulsion may be incubated on the same microfluidic device as was used to add the reagents and/or barcode, or may be incubated on a separate device. In certain embodiments, incubating the emulsion under conditions that facilitates nucleic acid amplification is performed on the same microfluidic device used to encapsulate the cells and lyse the cells. Incubating the emulsions may take a variety of forms. In certain aspects, the emulsions containing the reaction mix, barcode, and cell lysate may be flowed through a channel that incubates the emulsions under conditions effective for nucleic acid amplification. Flowing the microdroplets through a channel may involve a channel that snakes over various temperature zones maintained at temperatures effective for PCR. Such channels may, for example, cycle over two or more temperature zones, wherein at least one zone is maintained at about 65° C. and at least one zone is maintained at about 95° C. As the drops move through such zones, their temperature cycles, as needed for nucleic acid amplification. The number of zones, and the respective temperature of each zone, may be readily determined by those of skill in the art to achieve the desired nucleic acid amplification.

In various embodiments, following nucleic acid amplification, emulsions containing the amplified nucleic acids are collected. In various embodiments, the emulsions are collected in a well, such as a well of a microfluidic device. In various embodiments, the emulsions are collected in a reservoir or a tube, such as an Eppendorf tube. Once collected, the amplified nucleic acids across the different emulsions are pooled. In one embodiment, the emulsions are broken by providing an external stimuli to pool the amplified nucleic acids. In one embodiment, the emulsions naturally aggregate over time given the density differences between the aqueous phase and immiscible oil phase. Thus, the amplified nucleic acids pool in the aqueous phase.

In various embodiments, following pooling, the amplified nucleic acids can undergo further preparation for sequencing. For example, sequencing adapters can be added to the pooled nucleic acids. Example sequencing adapters are P5 and P7 sequencing adapters. The sequencing adapters enable the subsequent sequencing of the nucleic acids.

### Example Barcoding of Antibody-Conjugated Oligonucleotide and Genomic DNA

FIG. 4A illustrates the priming and barcoding of an antibody-conjugated oligonucleotide, in accordance with an embodiment. In various embodiments, the antibody-conjugated oligonucleotide can be specific for a surface protein. In various embodiments, the antibody-conjugated oligonucleotide can be specific for an intracellular protein. Specifically, FIG. 4A depicts step 410 involving the priming of the antibody oligonucleotide 304 and further depicts step 420 which involves the barcoding and amplification of the antibody oligonucleotide 304. In various embodiments, step 410 occurs within a first emulsion during which cell lysis occurs and step 420 occurs within a second emulsion during which cell barcoding and nucleic acid amplification occurs. In such embodiments, the primer 405 is provided in the reagents and the barcodes are provided via a barcode bead. In some embodiments, both steps 410 and 420 occur within the second emulsion.

The antibody oligonucleotide 304 is conjugated to an antibody. In various embodiments, an antibody oligonucleotide 304 includes a PCR handle, a tag sequence (e.g., an antibody tag), and a capture sequence that links the oligonucleotide to the antibody. In various embodiments, the antibody oligonucleotide 304 is conjugated to a region of the antibody, such that the antibody's ability to bind a target epitope is unaffected. For example, the antibody oligonucleotide 304 can be linked to a Fc region of the antibody, thereby leaving the variable regions of the antibody unaffected and available for epitope binding. In various the antibody oligonucleotide 304 can include a unique molecular identifier (UMI). In various embodiments, the UMI can be inserted before or after the antibody tag. In various embodiments, the UMI can flank either end of the antibody tag. In various embodiments, the UMI enables the quantification of the particular antibody oligonucleotide 304 and antibody combination.

In various embodiments, the antibody oligonucleotide 304 includes more than one PCR handle. For example, the antibody oligonucleotide 304 can include two PCR handles, one on each end of the antibody oligonucleotide 304. In various embodiments, one of the PCR handles of the antibody oligonucleotide 304 is conjugated to the antibody. Here, forward and reverse primers can be provided that hybridize with the two PCR handles, thereby enabling amplification of the antibody oligonucleotide 304.

Generally, the antibody tag of the antibody oligonucleotide 304 enables the subsequent identification of the antibody (and corresponding protein that the antibody specifically binds to). For example, the antibody tag can serve as an identifier e.g., a barcode for identifying the type of protein for which the antibody binds to. In various embodiments, antibodies that bind to the same target are each linked to the same antibody tag. For example antibodies that bind to the same epitope of a target protein are each linked to the same antibody tag, thereby enabling the subsequent determination of the presence of the target protein. In various embodiments, antibodies that bind different epitopes of the same target protein can be linked to the same antibody tag, thereby enabling the subsequent determination of the presence of the target protein.

In some embodiments, an oligonucleotide sequence is encoded by its nucleobase sequence and thus confers a combinatorial tag space far exceeding what is possible with conventional approaches using fluorescence. For example, a modest tag length of ten bases provides over a million unique sequences, sufficient to label an antibody against every epitope in the human proteome. Indeed, with this approach, the limit to multiplexing is not the availability of unique tag sequences but, rather, that of specific antibodies that can detect the epitopes of interest in a multiplexed reaction.

Step 410 depicts the priming of the antibody oligonucleotide 304 by a primer 405. As shown in FIG. 4A, the primer 405 may include a PCR handle and a common sequence. Here, the PCR handle of the primer 405 is complementary to the PCR handle of the antibody oligonucleotide 304. Thus, the primer 405 primes the antibody oligonucleotide 304 given the hybridization of the PCR handles. In various embodiments, extension occurs from the PCR handle of the antibody oligonucleotide 304 (as indicated by the dotted arrow). In various embodiments, extension occurs from the PCR handle of the primer 405, thereby generating a nucleic acid with the antibody tag and capture sequence.

Step 420 depicts the barcoding of the antibody oligonucleotide 304. As shown in FIG. 4, the barcode (e.g., cell barcode) is releasably attached to a bead and is further linked to a common sequence. Here, the common sequence linked to the cell barcode is complementary to the common sequence linked to the PCR handle, antibody tag, and capture sequence. The antibody oligonucleotide is extended to include the common sequence and cell barcode.

In various embodiments, the antibody oligonucleotide is amplified, thereby generating amplicons with the cell barcode, common sequence, PCR handle, antibody tag, and capture sequence. In various embodiments, the capture sequence contains a biotin oligonucleotide capture site, which enables streptavidin bead enrichment prior to library preparation. In various embodiments, the barcoded antibody-oligonucleotides can be enriched by size separation from the amplified genomic DNA targets.

FIG. 4B illustrates the priming and barcoding of genomic DNA 455, in accordance with an embodiment. Specifically, FIG. 4B depicts step 460 involving the priming of the genomic DNA 455 and further depicts step 470 which involves the barcoding and amplification of the genomic DNA 455. In various embodiments, step 460 occurs within a first emulsion during which cell lysis occurs and step 470 occurs within a second emulsion during which cell barcoding and nucleic acid amplification occurs. In such embodiments, the primer 465 is added in the reagents and the barcode and forward primers shown in step 470 are added. In some embodiments, step 460 and step 470 both occur within a single emulsion (e.g., a second emulsion) during which cell barcoding and nucleic acid amplification occurs. In such embodiments, the primer 465 shown in step 460 and the barcode and forward primers shown in step 470 are added.

At step 460, a primer 465 (as indicated by the dotted line) hybridizes with a portion of the genomic DNA 455. In various embodiments, the primer 465 is a gene specific primer that targets a sequence of a gene of interest. Therefore, the primer 465 hybridizes with a sequence of the genomic DNA 455 corresponding to the gene of interest. In various embodiments the primer 465 further includes a PCR handle or is linked to a PCR handle.

At step 470, a primer 475 (as indicated by the dotted line) hybridizes with a portion of the genomic DNA 455. In various embodiments, the primer 475 includes a PCR handle or is linked to a PCR handle. In various embodiments, the primer 475 is a gene specific primer that targets another sequence of the gene of interest that differs from the sequence targeted by the primer 465. Additionally, a cell barcode (cell BC), which is releasably attached to a bead, is linked to a PCR handle which hybridizes with the PCR handle of the forward primer. Nucleic acid amplification generates amplicons, each of which include the cell barcode, PCR handle, forward primer, the gene sequence of interest the primer 465, and the PCR handle.

### Sequencing and Read Alignment

Amplified nucleic acids (e.g., amplicons) are sequenced to obtain sequence reads for generating a sequencing library. Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. As an example, amplified nucleic acids may be sequenced on an Illumina MiSeq platform.

When pyrosequencing libraries of NGS fragments are cloned in-situ amplified by capture of one matrix molecule using granules coated with oligonucleotides complementary to adapters. Each granule containing a matrix of the same type is placed in a microbubble of the “water in oil” type and the matrix is cloned amplified using a method called emulsion PCR. After amplification, the emulsion is destroyed and the granules are stacked in separate wells of a titration picoplate acting as a flow cell during sequencing reactions. The ordered multiple administration of each of the four dNTP reagents into the flow cell occurs in the presence of sequencing enzymes and a luminescent reporter, such as luciferase. In the case where a suitable dNTP is added to the 3′ end of the sequencing primer, the resulting ATP produces a flash of luminescence within the well, which is recorded using a CCD camera. It is possible to achieve a read length of more than or equal to 400 bases, and it is possible to obtain 10⁶ readings of the sequence, resulting in up to 500 million base pairs (megabytes) of the sequence. Additional details for pyrosequencing are described in Voelkerding et al., Clinical Chem., 55: 641-658, 2009; MacLean et al., Nature Rev. Microbiol., 7: 287-296; U.S. Pat. Nos. 6,210,891; 6,258,568; each of which is hereby incorporated by reference in its entirety.

On the Solexa/Illumina platform, sequencing data is produced in the form of short readings. In this method, fragments of a library of NGS fragments are captured on the surface of a flow cell that is coated with oligonucleotide anchor molecules. An anchor molecule is used as a PCR primer, but due to the length of the matrix and its proximity to other nearby anchor oligonucleotides, elongation by PCR leads to the formation of a “vault” of the molecule with its hybridization with the neighboring anchor oligonucleotide and the formation of a bridging structure on the surface of the flow cell. These DNA loops are denatured and cleaved. Straight chains are then sequenced using reversibly stained terminators. The nucleotides included in the sequence are determined by detecting fluorescence after inclusion, where each fluorescent and blocking agent is removed prior to the next dNTP addition cycle. Additional details for sequencing using the Illumina platform are found in Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each of which is hereby incorporated by reference in its entirety.

Sequencing of nucleic acid molecules using SOLiD technology includes clonal amplification of the library of NGS fragments using emulsion PCR. After that, the granules containing the matrix are immobilized on the derivatized surface of the glass flow cell and annealed with a primer complementary to the adapter oligonucleotide. However, instead of using the indicated primer for 3′ extension, it is used to obtain a 5′ phosphate group for ligation for test probes containing two probe-specific bases followed by 6 degenerate bases and one of four fluorescent labels. In the SOLiD system, test probes have 16 possible combinations of two bases at the 3′ end of each probe and one of four fluorescent dyes at the 5′ end. The color of the fluorescent dye and, thus, the identity of each probe, corresponds to a certain color space coding scheme. After many cycles of alignment of the probe, ligation of the probe and detection of a fluorescent signal, denaturation followed by a second sequencing cycle using a primer that is shifted by one base compared to the original primer. In this way, the sequence of the matrix can be reconstructed by calculation; matrix bases are checked twice, which leads to increased accuracy. Additional details for sequencing using SOLiD technology are found in Voelkerding et al., Clinical Chem., 55: 641-658, 2009; MacLean et al., Nature Rev. Microbiol., 7: 287-296; U.S. Pat. Nos. 5,912,148; 6,130,073; each of which is incorporated by reference in its entirety.

In particular embodiments, HeliScope from Helicos BioSciences is used. Sequencing is achieved by the addition of polymerase and serial additions of fluorescently-labeled dNTP reagents. Switching on leads to the appearance of a fluorescent signal corresponding to dNTP, and the specified signal is captured by the CCD camera before each dNTP addition cycle. The reading length of the sequence varies from 25-50 nucleotides with a total yield exceeding 1 billion nucleotide pairs per analytical work cycle. Additional details for performing sequencing using HeliScope are found in Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each of which is incorporated by reference in its entirety.

In some embodiments, a Roche sequencing system 454 is used. Sequencing 454 involves two steps. In the first step, DNA is cut into fragments of approximately 300-800 base pairs, and these fragments have blunt ends. Oligonucleotide adapters are then ligated to the ends of the fragments. The adapter serves as primers for amplification and sequencing of fragments. Fragments can be attached to DNA-capture beads, for example, streptavidin-coated beads, using, for example, an adapter that contains a 5′-biotin tag. Fragments attached to the granules are amplified by PCR within the droplets of an oil-water emulsion. The result is multiple copies of cloned amplified DNA fragments on each bead. At the second stage, the granules are captured in wells (several picoliters in volume). Pyrosequencing is carried out on each DNA fragment in parallel. Adding one or more nucleotides leads to the generation of a light signal, which is recorded on the CCD camera of the sequencing instrument. The signal intensity is proportional to the number of nucleotides included. Pyrosequencing uses pyrophosphate (PPi), which is released upon the addition of a nucleotide. PPi is converted to ATP using ATP sulfurylase in the presence of adenosine 5′ phosphosulfate. Luciferase uses ATP to convert luciferin to oxyluciferin, and as a result of this reaction, light is generated that is detected and analyzed. Additional details for performing sequencing 454 are found in Margulies et al. (2005) Nature 437: 376-380, which is hereby incorporated by reference in its entirety.

Ion Torrent technology is a DNA sequencing method based on the detection of hydrogen ions that are released during DNA polymerization. The microwell contains a fragment of a library of NGS fragments to be sequenced. Under the microwell layer is the hypersensitive ion sensor ISFET. All layers are contained within a semiconductor CMOS chip, similar to the chip used in the electronics industry. When dNTP is incorporated into a growing complementary chain, a hydrogen ion is released that excites a hypersensitive ion sensor. If homopolymer repeats are present in the sequence of the template, multiple dNTP molecules will be included in one cycle. This results in a corresponding amount of hydrogen atoms being released and in proportion to a higher electrical signal. This technology is different from other sequencing technologies that do not use modified nucleotides or optical devices. Additional details for Ion Torrent Technology is found in Science 327 (5970): 1190 (2010); US Patent Application Publication Nos. 20090026082, 20090127589, 20100301398, 20100197507, 20100188073, and 20100137143, each of which is incorporated by reference in its entirety.

In various embodiments, sequencing reads obtained from the NGS methods can be filtered by quality and grouped by barcode sequence using any algorithms known in the art, e.g., Python script barcodeCleanup.py. In some embodiments, a given sequencing read may be discarded if more than about 20% of its bases have a quality score (Q-score) less than Q20, indicating a base call accuracy of about 99%. In some embodiments, a given sequencing read may be discarded if more than about 5%, about 10%, about 15%, about 20%, about 25%, about 30% have a Q-score less than Q10, Q20, Q30, Q40, Q50, Q60, or more, indicating a base call accuracy of about 90%, about 99%, about 99.9%, about 99.99%, about 99.999%, about 99.9999%, or more, respectively.

In some embodiments, sequencing reads associated with a barcode containing less than 50 reads may be discarded to ensure that all barcode groups, representing single cells, contain a sufficient number of high-quality reads. In some embodiments, all sequencing reads associated with a barcode containing less than 30, less than 40, less than 50, less than 60, less than 70, less than 80, less than 90, less than 100 or more may be discarded to ensure the quality of the barcode groups representing single cells.

In various embodiments, sequence reads with common barcode sequences (e.g., meaning that sequence reads originated from the same cell) may be aligned to a reference genome using known methods in the art to determine alignment position information. For example, sequence reads derived from genomic DNA can be aligned to a range of positions of a reference genome. In various embodiments, sequence reads derived from genomic DNA can align with a range of positions corresponding to a gene of the reference genome. The alignment position information may indicate a beginning position and an end position of a region in the reference genome that corresponds to a beginning nucleotide base and end nucleotide base of a given sequence read. A region in the reference genome may be associated with a target gene or a segment of a gene. Further details for aligning sequence reads to reference sequences is described in U.S. application Ser. No. 16/279,315, which is hereby incorporated by reference in its entirety. In various embodiments, an output file having SAM (sequence alignment map) format or BAM (binary alignment map) format may be generated and output for subsequent analysis, such as for determining cell trajectory.

Cells and Cell Populations

Embodiments described herein involve the single-cell analysis of cells. In various embodiments, the cells are healthy cells. In various embodiments, the cells are diseased cells. Examples of diseased cells include cancer cells, such as cells of hematologic malignancies or solid tumors. Examples of hematologic malignancies include, but are not limited to, acute lymphoblastic leukemia, acute myeloid leukemia, chronic lymphocytic leukemia, chronic myeloid leukemia, classic Hodgkin's Lymphoma, diffuse large B-cell lymphoma, follicular lymphoma, mantle cell lymphoma, multiple myeloma, myelodysplastic syndromes, myeloid, myeloproliferative neoplasms, or T-cell lymphoma. Examples of solid tumors include, but are not limited to, breast invasive carcinoma, colon adenocarcinoma, glioblastoma multiforme, kidney renal clear cell carcinoma, liver hepatocellular carcinoma, lung adenocarcinoma, lung squamous cell carcinoma, ovarian cancer, pancreatic adenocarcinoma, prostate adenocarcinoma, or skin cutaneous melanoma.

In various embodiments, the single-cell analysis is performed on a population of cells. The population of cells can be a heterogeneous population of cells. In one embodiment, the population of cells can include both cancerous and non-cancerous cells. In one embodiment, the population of cells can include cancerous cells that are heterogenous amongst themselves. In various embodiments, the population of cells can be obtained from a subject. For example, a sample is taken from a subject, and the population of cells in the sample are isolated for performing single-cell analysis.

Barcodes and Barcoded Beads

Embodiments of the invention involve providing one or more barcode sequences for labeling analytes of a single cell during step 170 shown in FIG. 1B. The one or more barcode sequences are encapsulated in an emulsion with a cell lysate derived from a single cell. As such, the one or more barcodes label analytes of the cell, thereby enabling the subsequent determination that sequence reads derived from the analytes originated from the same single cell.

In various embodiments, a plurality of barcodes are added to a droplet with a cell lysate. In various embodiments, the plurality of barcodes added to a droplet includes at least 10², at least 10³, at least 10⁴, at least 10⁵, at least 10⁵, at least 10⁶, at least 10⁷, or at least 10⁸ barcodes. In various embodiments, the plurality of barcodes added to an emulsion have the same barcode sequence. For example, multiple copies of the same barcode label are added to an emulsion to label multiple analytes derived from the cell lysate, thereby enabling identification of the cell from which an analyte originates from. In various embodiments, the plurality of barcodes added to an emulsion comprise a ‘unique identification sequence’ (UMI). A UMI is a nucleic acid having a sequence which can be used to identify and/or distinguish one or more first molecules to which the UMI is conjugated from one or more second molecules to which a distinct UMI, having a different sequence, is conjugated. UMIs are typically short, e.g., about 5 to 20 bases in length, and may be conjugated to one or more target molecules of interest or amplification products thereof. UMIs may be single or double stranded. In some embodiments, both a barcode sequence and a UMI are incorporated into a barcode. Generally, a UMI is used to distinguish between molecules of a similar type within a population or group, whereas a barcode sequence is used to distinguish between populations or groups of molecules that are derived from different cells. In some embodiments, where both a UMI and a barcode sequence are utilized, the UMI is shorter in sequence length than the barcode sequence. The use of barcodes is further described in U.S. patent application Ser. No. 15/940,850, which is hereby incorporated by reference in its entirety.

In some embodiments, the barcodes are single-stranded barcodes. Single-stranded barcodes can be generated using a number of techniques. For example, they can be generated by obtaining a plurality of DNA barcode molecules in which the sequences of the different molecules are at least partially different. These molecules can then be amplified so as to produce single stranded copies using, for instance, asymmetric PCR. Alternatively, the barcode molecules can be circularized and then subjected to rolling circle amplification. This will yield a product molecule in which the original DNA barcoded is concatenated numerous times as a single long molecule.

In some embodiments, circular barcode DNA containing a barcode sequence flanked by any number of constant sequences can be obtained by circularizing linear DNA. Primers that anneal to any constant sequence can initiate rolling circle amplification by the use of a strand displacing polymerase (such as Phi29 polymerase), generating long linear concatemers of barcode DNA.

In various embodiments, barcodes can be linked to a primer sequence that enables the barcode to label a target nucleic acid. In one embodiment, the barcode is linked to a forward primer sequence. In various embodiments, the forward primer sequence is a gene specific primer that hybridizes with a forward target of a nucleic acid. In various embodiments, the forward primer sequence is a constant region, such as a PCR handle, that hybridizes with a complementary sequence attached to a gene specific primer. The complementary sequence attached to a gene specific primer can be provided. Including a constant forward primer sequence on barcodes may be preferable as the barcodes can have the same forward primer and need not be individually designed to be linked to gene specific forward primers.

In various embodiments, barcodes can be releasably attached to a support structure, such as a bead. Therefore, a single bead with multiple copies of barcodes can be partitioned into an emulsion with a cell lysate, thereby enabling labeling of analytes of the cell lysate with the barcodes of the bead. Example beads include solid beads (e.g., silica beads), polymeric beads, or hydrogel beads (e.g., polyacrylamide, agarose, or alginate beads). Beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-split cycle. Additional details of example beads and their synthesis is described in International Application No. PCT/US2016/016444, which is hereby incorporated by reference in its entirety.

Reagents

Embodiments described herein include the encapsulation of a cell with reagents (e.g., reagents 120A and/or 120B in FIG. 1B) within a droplet (e.g., a first droplet and/or a second droplet in FIG. 1B). Generally, the reagents interact with the encapsulated cell under conditions in which the cell is lysed, thereby releasing target analytes of the cell. The reagents can further interact with target analytes to prepare for subsequent barcoding and/or amplification.

In various embodiments, the reagents include one or more lysing agents that cause the cell to lyse. Examples of lysing agents include detergents such as Triton X-100, Nonidet P-40 (NP40) as well as cytotoxins. In some embodiments, the reagents include NP40 detergent which is sufficient to disrupt the cell membrane and cause cell lysis, but does not disrupt chromatin-packaged DNA. In various embodiments, the reagents include 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% NP40 (v/v). In various embodiments, the reagents include at least at least 0.01%, at least 0.05%, 0.1%, at least 0.5%, at least 1%, at least 2%, at least 3%, at least 4%, or at least 5% NP40 (v/v).

In various embodiments, the reagents further include proteases that assist in the lysing of the cell and/or accessing of genomic DNA. Examples of proteases include proteinase K, pepsin, protease-subtilisin Carlsberg, protease type X-Bacillus thermoproteolyticus, protease type XIII—Aspergillus saitoi. In various embodiments, the reagents includes 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 6.0 mg/mL, 7.0 mg/mL, 8.0 mg/mL, 9.0 mg/mL, or 10.0 mg/mL of proteases. In various embodiments, the reagents include between 0.1 mg/mL and 5 mg/mL of proteases. In various embodiments, the reagents include between 0.5 mg/mL and 2.5 mg/mL of proteases. In various embodiments, the reagents include between 0.75 mg/mL and 1.5 mg/mL of proteases. In various embodiments, the reagents include between 0.9 mg/mL and 1.1 mg/mL of proteases.

In various embodiments, the reagents can further include dNTPs, stabilization agents such as dithothreitol (DTT), and buffer solutions. In various embodiments, the reagents can include primers, such as antibody tag primers. In various embodiments, the reagents can include primers, such as reverse primers that hybridize with a target analyte (e.g., genomic DNA or an antibody oligonucleotide). In various embodiments, such primers can be gene specific primers. Example primers are described in further detail below.

Primers (or Primer Reagents)

Embodiments of the invention described herein use primers to conduct the single-cell analysis. For example, primers are implemented during the workflow process shown in FIG. 1. Primers can be used to prime (e.g., hybridize) with specific sequences of nucleic acids of interest, such that the nucleic acids of interest can be barcoded and/or amplified. Specifically, primers hybridize to a target sequence and act as a substrate for enzymes (e.g., polymerases) that catalyze nucleic acid synthesis off a template strand to which the primer has hybridized. As described hereafter, primers can be provided in the workflow process shown in FIG. 1 in various steps. Referring again to FIG. 1, in various embodiments, primers can be included in the reagents 120 that are encapsulated with the cell 102. In various embodiments, primers can be included in the reagents that is encapsulated with the cell lysate 130. In various embodiments, primers can be included in or linked with a barcode 145 that is encapsulated with the cell lysate 130. Further description and examples of primers that are used in a single-cell analysis workflow process is described in U.S. application Ser. No. 16/749,731, which is hereby incorporated by reference in its entirety.

In various embodiments, the number of distinct primers in any of the reagents, or with barcodes may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

For targeted DNA sequencing primers in the reagents (e.g., reagents 120 in FIG. 1) may include reverse primers that are complementary to a reverse target sequence on a nucleic acid of interest (e.g., DNA or RNA). In various embodiments, primers in the reagents may be gene-specific primers that target a reverse target sequence of a gene of interest. In various embodiments, primers in the reagents may include forward primers that are complementary to a forward target sequence on a nucleic acid of interest (e.g., DNA). In various embodiments, primers in the reagents may be gene-specific primers that target a forward target of a gene of interest. In various embodiments, primers of the reagents form primer sets (e.g., forward primer and reverse primer) for a region of interest on a nucleic acid. Example gene-specific primers can be primers that target any of the genes identified in the “Targeted Panels” section above.

The number of distinct forward or reverse primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

In various embodiments, instead of the primers being included in the reagents such primers can be included or linked to a barcode. In particular embodiments, the primers are linked to an end of the barcode and therefore, are available to hybridize with target sequences of nucleic acids in the cell lysate.

In various embodiments, primers of the reagents, or primers of barcodes may be added to an emulsion in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the primers of the reagents may be added in a separate step from the addition of a lysing agent (e.g., as exemplified in the two step workflow process shown in FIG. 1).

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that is substantially complementary or substantially identical to a corresponding target sequence in the sample, and each primer pair having a different corresponding target sequence. Accordingly, certain methods herein are used to detect or identify multiple target sequences from a single cell sample.

Example Kit Embodiments

Also provided herein are kits for performing the single-cell workflow for determining cellular genotypes and phenotypes of populations of cells. The kits may include one or more of the following: fluids for forming emulsions (e.g., carrier phase, aqueous phase), barcoded beads, micro fluidic devices for processing single cells, reagents for lysing cells and releasing cell analytes, reagents and buffers for labeling cells with antibodies, reaction mixtures for performing nucleic acid amplification reactions, and instructions for using any of the kit components according to the methods described herein.

System and/or Computer Embodiments

Additionally described herein are systems and computer embodiments for performing the single cell analysis described above. An example system can include a single cell workflow device and a computing device, such as single cell workflow device 106 and computing device 108 shown in FIG. 1A. In various embodiments, the single cell workflow device 106 is configured to perform the steps of cell encapsulation 160, lysis and digestion 165, cell re-encapsulation 170, and/or barcoding and amplification 175. In various embodiments, the computing device 108 is configured to perform the in silico steps such as read alignment, determining presence or absence of the analyte of the cell 260, and/or determining one or more mutations (e.g., SNV, indel, CNV etc).

In various embodiments, a single cell workflow device 106 includes at least a microfluidic device that is configured to encapsulate cells with reagents, encapsulate cell lysates with reagents, and perform nucleic acid amplification reactions. For example, the microfluidic device can include one or more fluidic channels that are fluidically connected. Therefore, the combining of an aqueous fluid through a first channel and a carrier fluid through a second channel results in the generation of emulsion droplets. In various embodiments, the fluidic channels of the microfluidic device may have at least one cross-sectional dimension on the order of a millimeter or smaller (e.g., less than or equal to about 1 millimeter). Additional details of microchannel design and dimensions is described in International Patent Application No. PCT/US2016/016444 and U.S. patent application Ser. No. 14/420,646, each of which is hereby incorporated by reference in its entirety. An example of a microfluidic device is the Tapestri™ Platform.

In various embodiments, the single cell workflow device 106 may also include one or more of: (a) a temperature control module for controlling the temperature of one or more portions of the subject devices and/or droplets therein and which is operably connected to the microfluidic device(s), (b) a detection module, i.e., a detector, e.g., an optical imager, operably connected to the microfluidic device(s), (c) an incubator, e.g., a cell incubator, operably connected to the microfluidic device(s), and (d) a sequencer operably connected to the microfluidic device(s). The one or more temperature and/or pressure control modules provide control over the temperature and/or pressure of a carrier fluid in one or more flow channels of a device. As an example, a temperature control module may be one or more thermal cycler that regulates the temperature for performing nucleic acid amplification. The one or more detection modules i.e., a detector, e.g., an optical imager, are configured for detecting the presence of one or more droplets, or one or more characteristics thereof, including their composition. In some embodiments, detector modules are configured to recognize one or more components of one or more droplets, in one or more flow channel. The sequencer is a hardware device configured to perform sequencing, such as next generation sequencing. Examples of sequencers include Illumina sequencers (e.g., MiniSeg™, MiSeg™, NextSeg™ 550 Series, or NextSeg™ 2000), Roche sequencing system 454, and Thermo Fisher Scientific sequencers (e.g., Ion GeneStudio S5 system, Ion Torrent Genexus System).

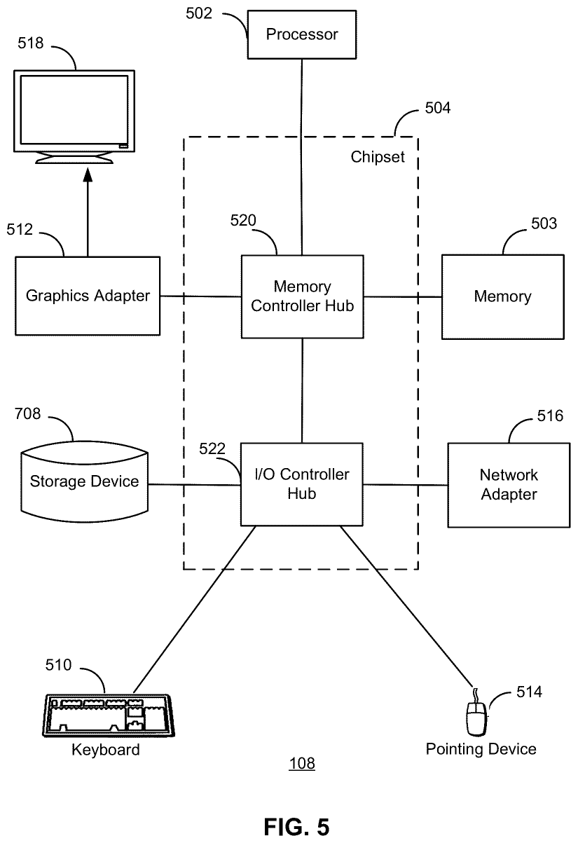

FIG. 5 depicts an example computing device for implementing system and methods described in reference to FIGS. 1-4B. For example, the example computing device 108 is configured to perform the in silico steps such as read alignment, determining presence or absence of the analyte of the cell 260, and/or determining one or more mutations (e.g., SNV, indel, CNV etc). Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

FIG. 5 illustrates an example computing device 108 for implementing system and methods described in FIGS. 1-4B. In some embodiments, the computing device 108 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, an input interface 514, and network adapter 516 are coupled to the I/O controller hub 522. Other embodiments of the computing device 108 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input interface 514 is a touch-screen interface, a mouse, track ball, or other type of input interface, a keyboard, or some combination thereof, and is used to input data into the computing device 108. In some embodiments, the computing device 108 may be configured to receive input (e.g., commands) from the input interface 514 via gestures from the user. The graphics adapter 512 displays images and other information on the display 518. For example, the display 518 can show an indication of a predicted cell trajectory. The network adapter 516 couples the computing device 108 to one or more computer networks.

The computing device 108 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502.

The types of computing devices 108 can vary from the embodiments described herein. For example, the computing device 108 can lack some of the components described above, such as graphics adapters 512, input interface 514, and displays 518. In some embodiments, a computing device 108 can include a processor 502 for executing instructions stored on a memory 506.

In various embodiments, methods described herein, such as methods of aligning sequence reads, methods of determining cellular genotypes and phenotypes, and/or methods of analyzing cells using cellular genotypes and phenotypes can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a cell trajectory of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### EXAMPLES

### Example 1: Examples Protocols for Analyzing Surface and Intracellular Proteins

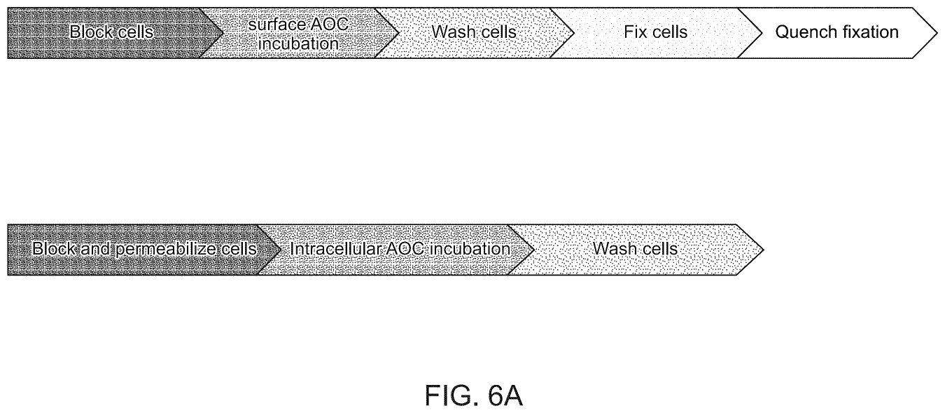

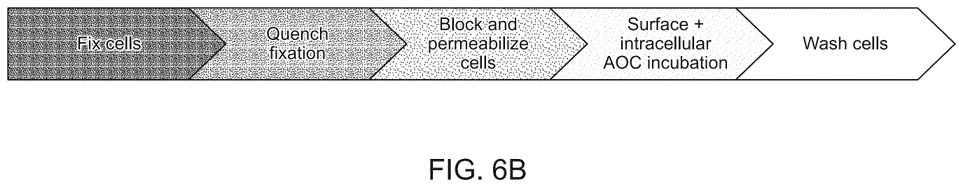

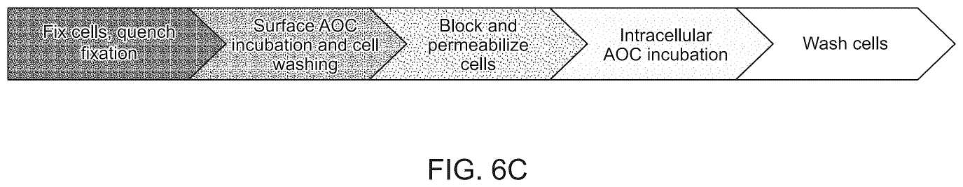

FIGS. 6A-6C show example protocols for surface and intracellular protein workflows.

Specifically, FIG. 6A is a flow process in which surface antibody-oligonucleotide conjugates and intracellular antibody-oligonucleotide conjugates are provided to cells separately. The flow process as shown in FIG. 6A included providing one or more surface antibody-oligonucleotide conjugates to the cell prior to fixing and permeabilizing the cell, wherein the one or more surface antibody-oligonucleotide conjugates are bound to the analyte located on the surface of the cell.

FIG. 6B is a flow process in which surface antibody-oligonucleotide conjugates and intracellular antibody-oligonucleotide conjugates are provided to cells in a single step. For example, the flow process shown in FIG. 6B included permeabilizing the cell prior to providing both surface antibody-oligonucleotide conjugates and intracellular antibody-oligonucleotide conjugates to the cell.

FIG. 6C is a flow process in which surface antibody-oligonucleotide conjugates and intracellular antibody-oligonucleotide conjugates are provided to cells separately. The flow process as shown in FIG. 6C included providing one or more surface antibody-oligonucleotide conjugates to the cell prior to permeabilizing the cell, wherein the one or more surface antibody-oligonucleotide conjugates are bound to the analyte located on the surface of the cell.

Referring to FIGS. 6A-6C, in some scenarios, example reagents for the step of “block cells” included Fc receptor blocker(s), and/or internal blocking reagent(s) (e.g., Prionex gelatin, salmon sperm DNA, mouse IgG, and/or EDTA). In some scenarios, example reagents for the step of “wash cells” included Dulbecco's phosphate-buffered saline (DPBS), fetal bovine serum (FBS), and/or a cell staining buffer. In some scenarios, example reagents (e.g. fixatives) for the step of “fix cells” included dithiobis(succinimidyl propionate) (DSP) and/or succinimidyl 3-(2-pyridyldithio)propionate (SPDP) in DPBS. In some scenarios, example reagents for the step of “quench fixation” included Tris-hydrochloride (Tris-HCL) and/or sodium chloride (NaCl). In some scenarios, example reagents for “permeabilize cells” included Triton X-100.

### Example 2: Single Cell Analysis Based on Genomic DNA, Intracellular Proteins, and Surface Proteins

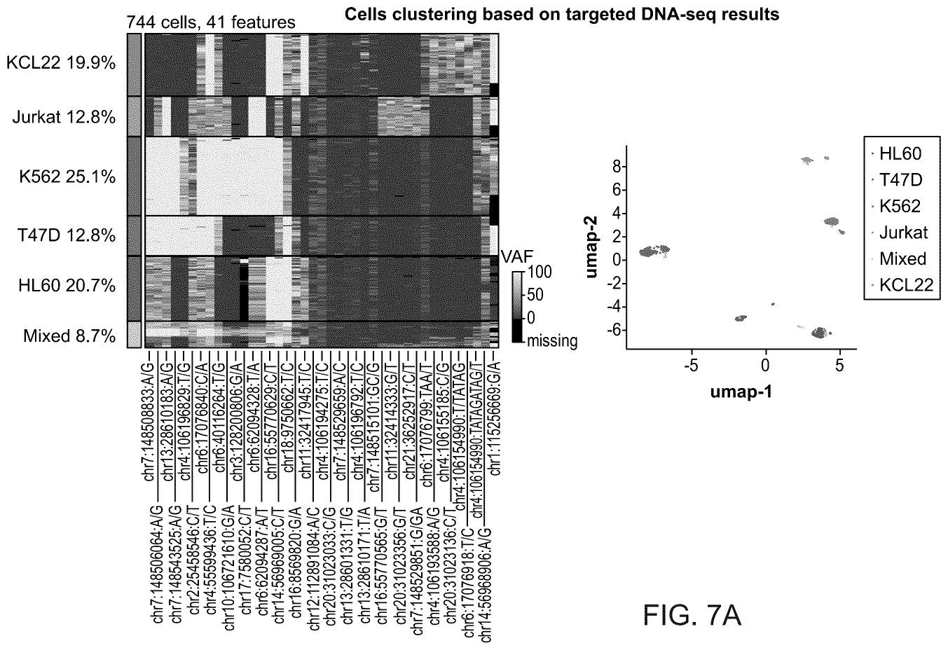

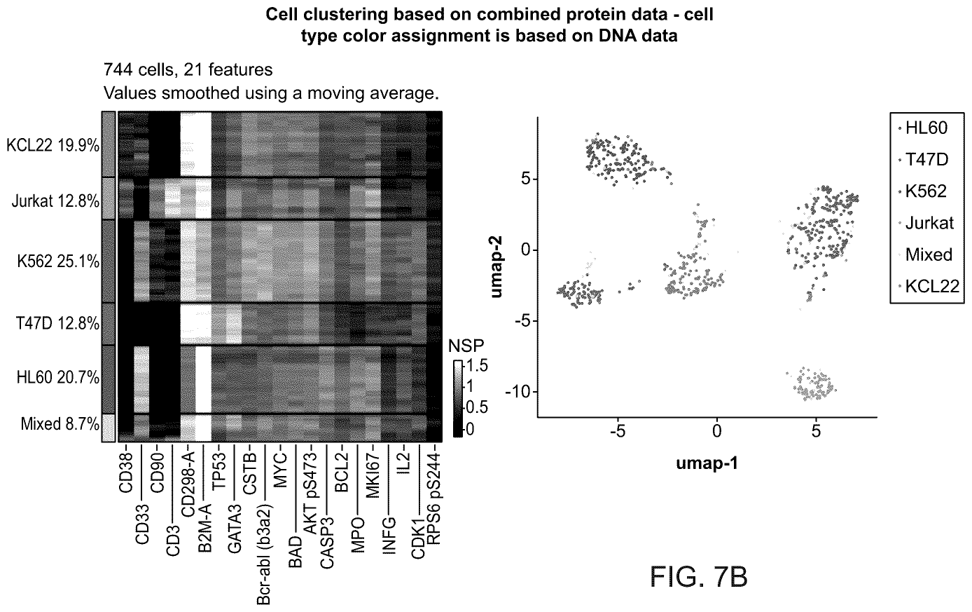

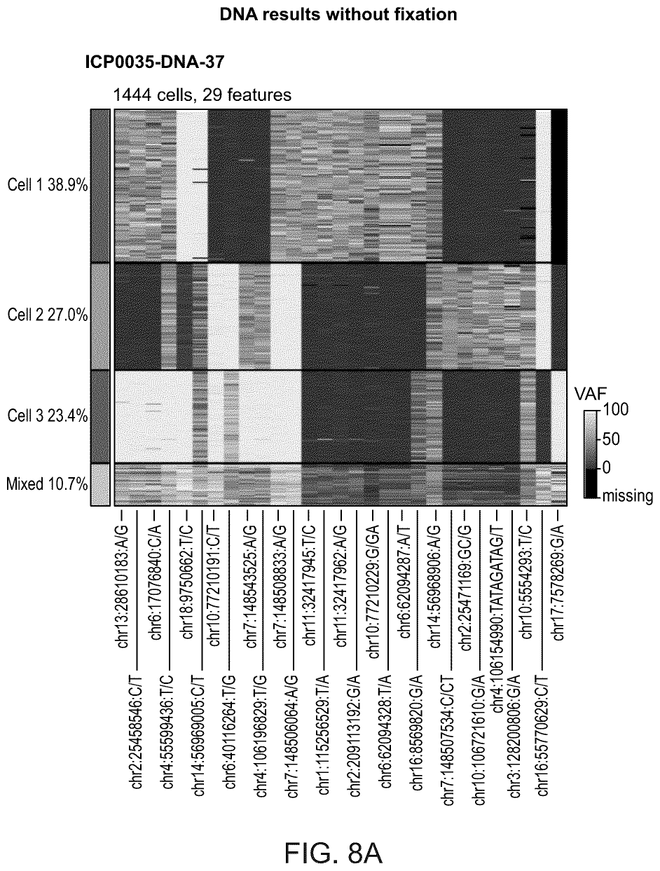

FIG. 7A illustrates example results of cell clustering based on targeted DNA-sequence data using the methods, systems, and apparatuses as described above. As shown in FIG. 7A, different clusters of cells with varying targeted DNA-seq results were identified, representing one out of a mixture of five cell lines including HL60, T47D, K562, Jurkat, and/or KCL22 cells in the analysis of 744 cells.

More specifically, a mixture of five cell lines, HL60, Jurkat, K562, KCL22, and T47D was prepared and washed twice in Dulbecco's phosphate-buffered saline (DPBS). The cells were blocked with a blocking buffer and then incubated with a panel of cell surface protein antibody-oligonucleotide conjugates. The surface protein targets in this panel included CD3, CD33, CD38, CD90, CD298, and B2M. After 30 minutes at room temperature (e.g., around 22° C.), the cells were washed in a wash buffer (e.g., DPBS+1% FBS) for four times and for 5 minutes each time. The cells were then fixed for 90 minutes, quenched for 10 minutes, and permeabilized and blocked for 30 minutes, prior to incubating with intracellular protein antibody-oligonucleotide conjugates at 4° C. for 17 hours. The intracellular protein targets in this panel included BAD, BCL2, MYC, IL2, GATA3, MKI67, MPO, TP53, CASP3, CSTB, CDK1, INFG, BCR-ABL, phospho-AKT, and phospho-RPS6. Resulting cells were washed in a wash buffer four times and for 5 minutes each time. The treated cells were then loaded onto Tapestri, where single-cell DNA and protein libraries were generated following the Tapestri protocol. The libraries were sequenced on the NextSeq 550 sequencer.

FIG. 7B illustrates example results of cell clustering based on combined protein data as described above. As shown in FIG. 7B, different clusters of cells with varying combined protein data (e.g., surface protein and intracellular protein targets were identified, representing one or mixture of five cell lines including HL60, T47D, K562, Jurkat, and/or KCL22 cells in the analysis of 744 cells. In some scenarios, the surface protein targets included CD3, CD33, CD38, CD90, CD298, and B2M, and the intracellular protein targets included BAD, BCL2, MYC, IL2, GATA3, MKI67, MPO, TP53, CASP3, CSTB, CDK1, INFG, BCR-ABL, phospho-AKT, and phospho-RPS6. In some scenarios, adding the detection of cell surface proteins enabled the examining of cell surface markers for cell typing and/or other surface proteins of interest (e.g., cell surface receptors).

As shown in FIGS. 7A and 7B, the methods described above can be used to simultaneously measure surface analytes, intracellular analytes, and genomic DNA from the same single cells. Thus, the methods described above can be potentially combined with RNA fusion detection, RNA expression, and/or other omics to obtain more information from single cells. Furthermore, detecting intracellular analytes might enable the study of biologically significant functional proteins (e.g., transcription factors, signal transduction proteins) that localize within the cytoplasm and nucleus.

As described herein in Example 2, in some scenarios, a minimum of 1 million cells were required for running a intracellular protein workflow because of extra steps such as fixation and/or permeabilization of cells) in the intracellular protein workflow. In some scenarios, a large cell number may be needed depending on the cell types (e.g. fragile cells).

The methods included in Example 2 were tested for DNA panels including 100 s-300 s amplicons, and protein panels including up to 22 AOCs. In the workflow as described in Example 2, a time consuming step was the titration of AOC panel to optimize the concentration for each AOC. A larger panel required more experiments for this step and a longer lead time before the panel was ready for use.

### Example 3: Single Cell Analysis Based on Genomic DNA and Intracellular Proteins

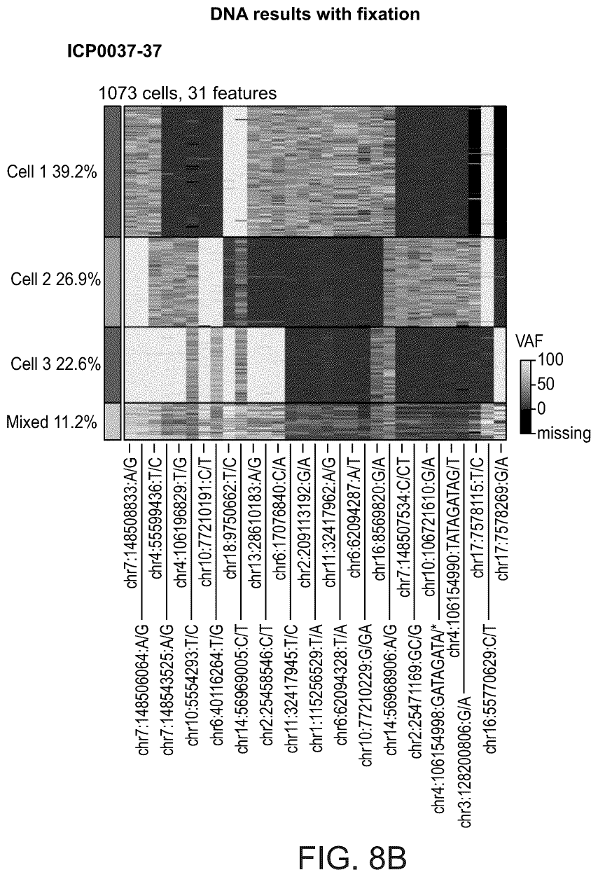

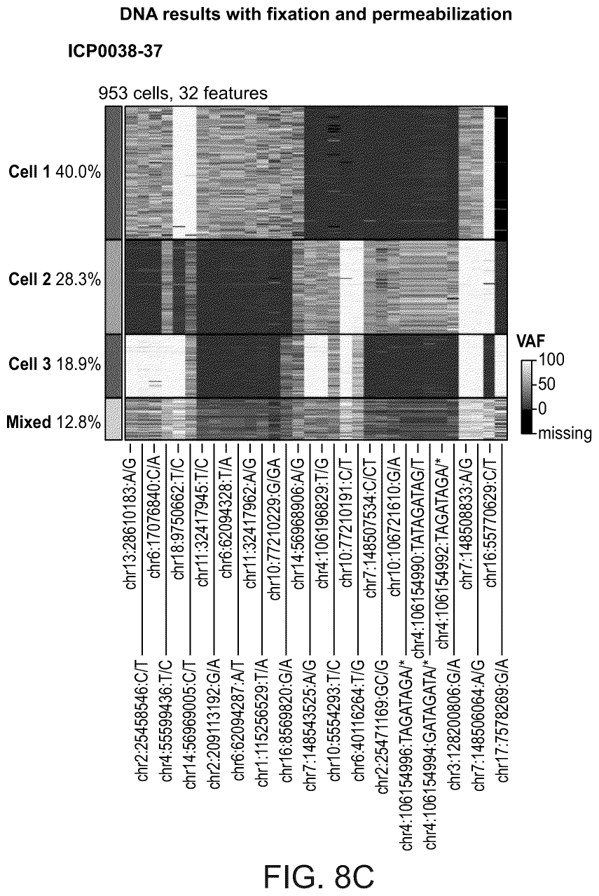

FIGS. 8A-8C illustrate example “DNA results” from workflows with and without steps of fixation and/or permeabilization of cells. FIG. 8A illustrates example DNA results without fixation or permeabilization of cells. FIG. 8B illustrates example DNA results with fixation and without permeabilization of cells. FIG. 8C illustrates example DNA results with fixation and permeabilization of cells. DNA libraries were generated from a mixture of three cell lines using the acute myeloid leukemia (AML) panel for the three experiments. Single nucleotide variants (SNV) were determined for each DNA target and the cells were clustered based on their unique SNV signatures. As shown in FIGS. 8A-8C, the cells were grouped into three clusters of similar proportion in the three conditions, indicating that the fixation and/or permeabilization of cells in the workflow did not compromise the DNA results.

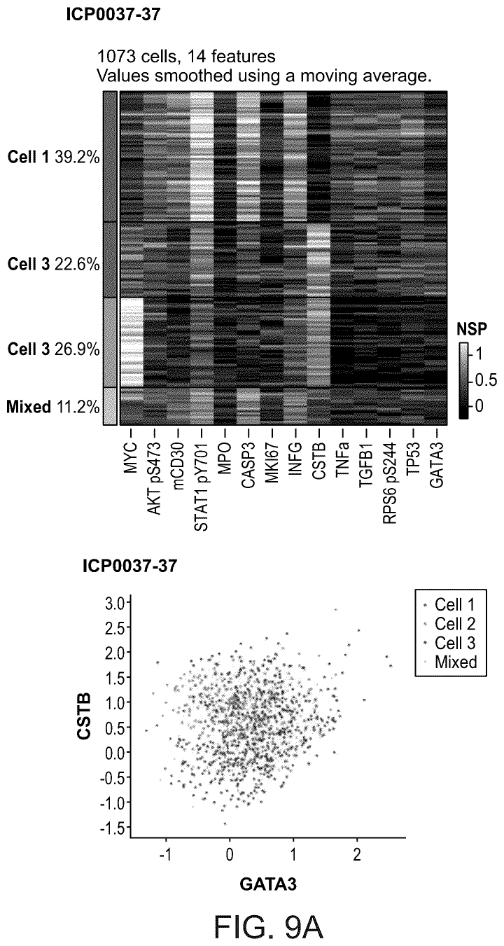

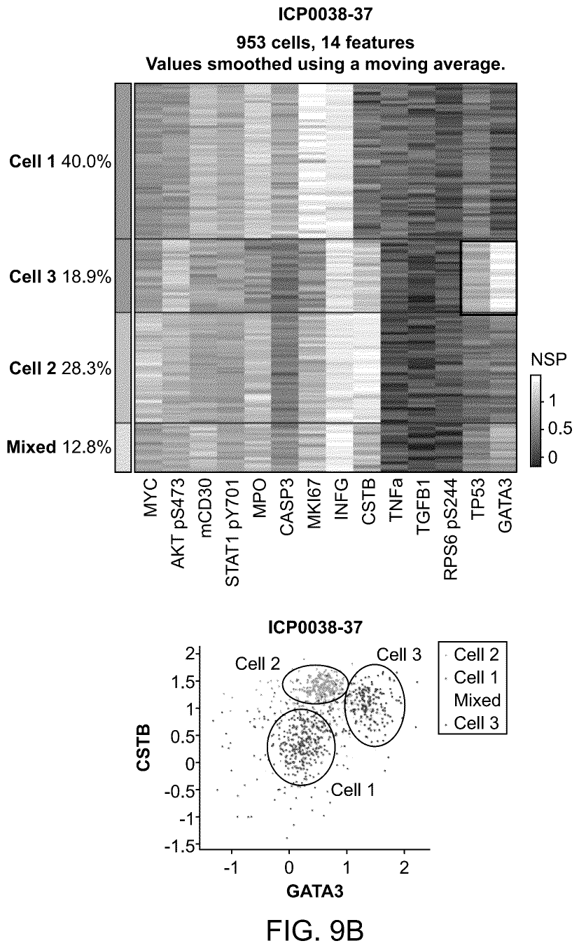

FIGS. 9A and 9B illustrate example protein results from workflows with steps of fixation and/or permeabilization. FIG. 9A illustrates example protein results with fixation and without permeabilization of cells. The intracellular proteins include MYC, AKT pS473, mCD30, STAT1 pY701, MPO, CASP3, MKI67, INFG, CSTB, TNFa, TGFB1, RPS6 pS244, TP53, and GATA3. FIG. 9B illustrates example protein results with fixation and permeabilization of cells. A comparison between protein results between FIGS. 9A and 9B shows that nuclear proteins including TB53 and GATA3 were detected in one of the cell lines (e.g., T47D cells) from the workflow that includes cell fixation and permeabilization steps (FIG. 9B), but nuclear proteins were not detected in the workflow without the cell permeabilization step (FIG. 9A). Thus, cell permeabilization likely enabled improved detection of nuclear proteins. As also shown in FIGS. 9A and 9B, some degree of intracellular protein detection was found, because cell fixation could slightly permeabilize the cell. Thus, adding a permeabilization step provides more accurate intracellular protein results. Furthermore, the bottom panel of FIG. 9B depicts “UMAP separation” of cell clusters based on protein expression, which is improved with cell permeabilization.

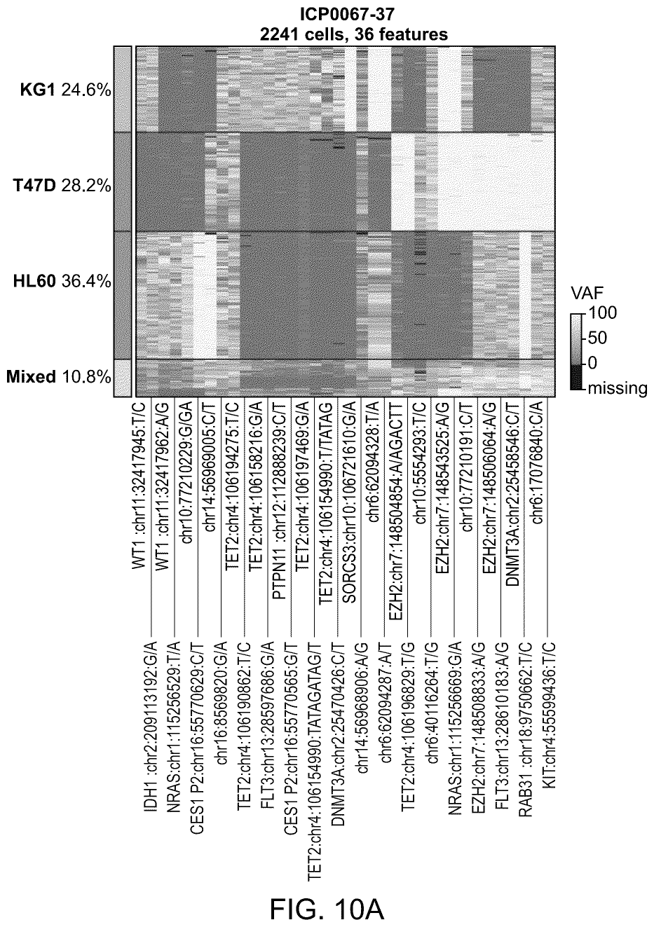

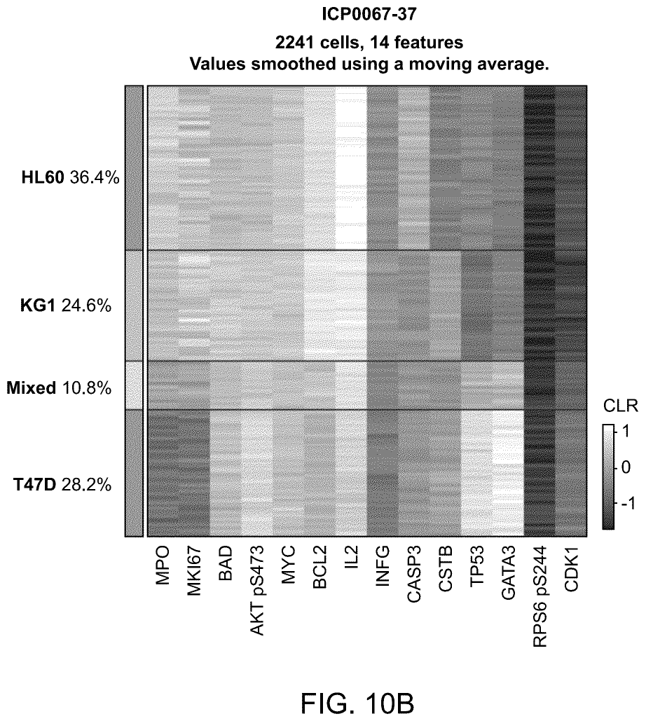

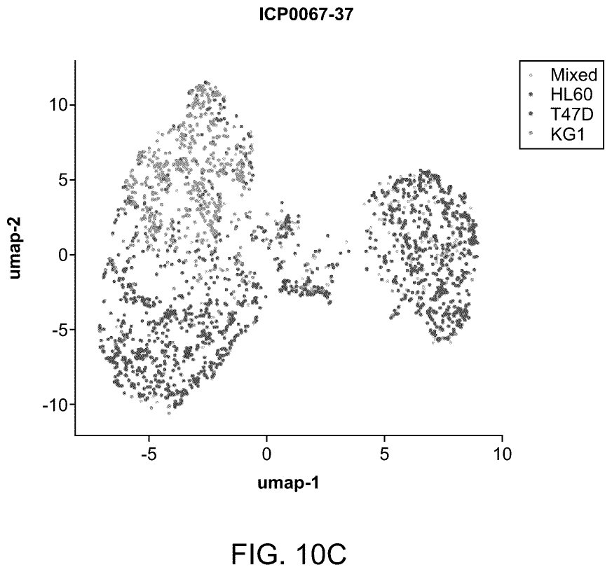

FIGS. 10A-10C illustrate results with cell lines HL60, T47D, KG1, and their mixture thereof. The results in FIG. 10A-10C involved acute myeloid leukemia (AML) panel including 138 amplicons and 14 antibody-oligonucleotide conjugates (ADCs). In particular, as shown on the x-axis of FIG. 10B, the intracellular proteins of the protein panel include MPO, MKI67, BAD, AKT pS473, MYC, BCL2, IL2, INFG, CASP3, CSTB, TP53, GATA3, RPS6 pS244, and CDK1.

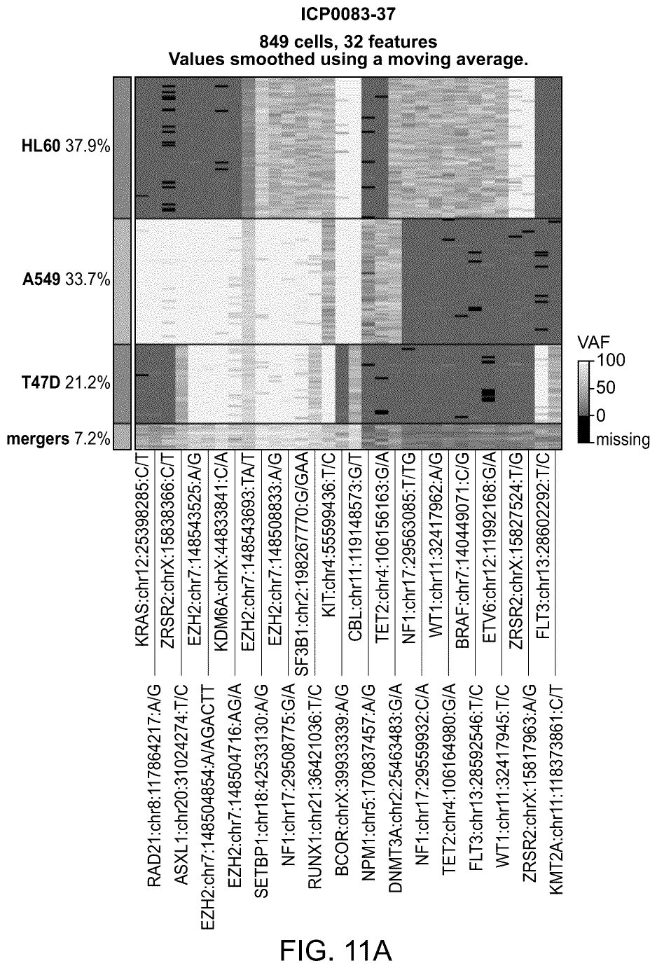

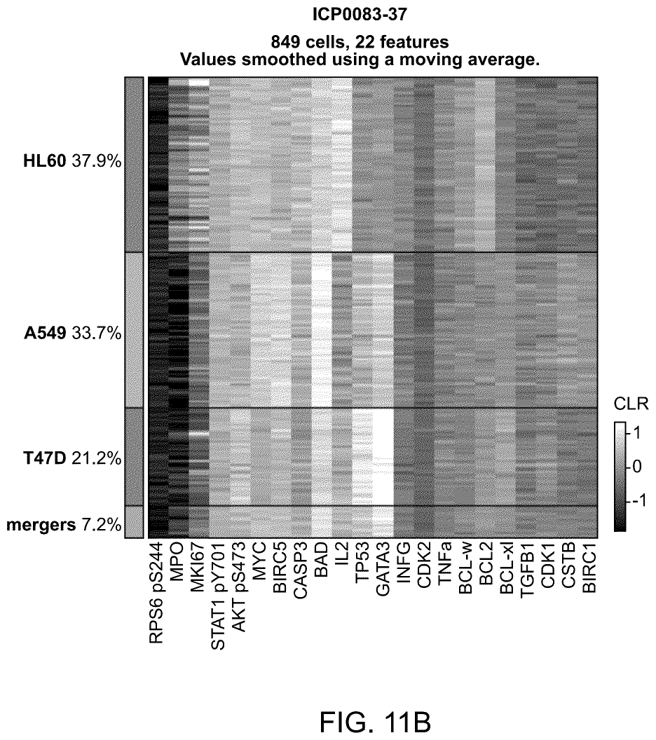

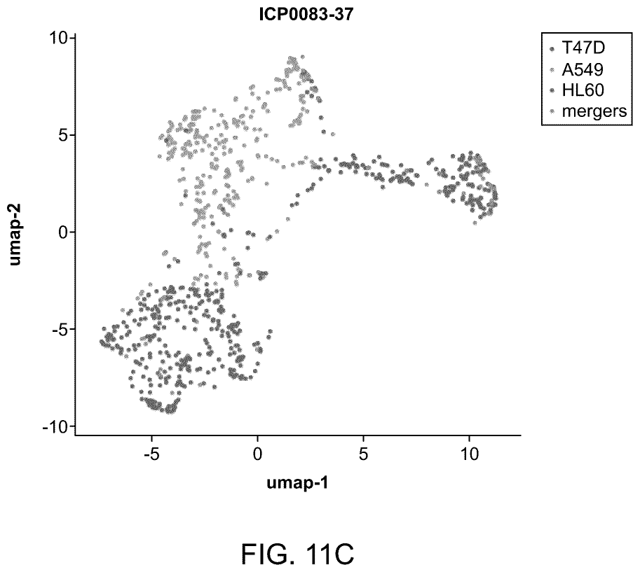

FIGS. 11A-11C illustrate results with cell lines T47D, A549, HL60, and their mergers thereof. The results in FIG. 11A-11C involved myeloid (MYE) panel including 312 amplicons and 22 antibody-oligonucleotide conjugates (ADCs). In particular, as shown on the x-axis of FIG. 11B, the intracellular proteins of the protein panel include RPS6 pS244, MPO, MKI67, STAT1 pY701, AKT pS473, MYC, BIRC5, CASP3, BAD, IL2, TP53, GATA3, IFNG, CDK2, TNFa, BCL-w, BCL2, BCL-xl, TGFB1, CDK1, CSTB, and BIRC1.

Altogether, the results shown in FIGS. 10A-10C and 11A-11C show that cell lines can be successfully distinguished based on DNA amplicons and expression of specific intracellular proteins.

### Example 4: Single Cell Analysis Based on SNV, CNV, and Surface Proteins

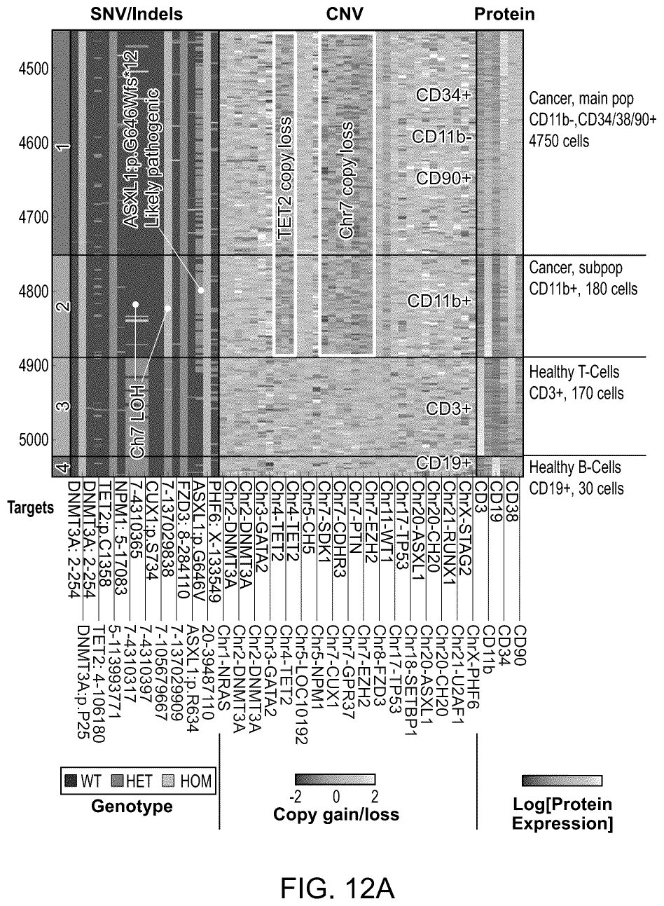

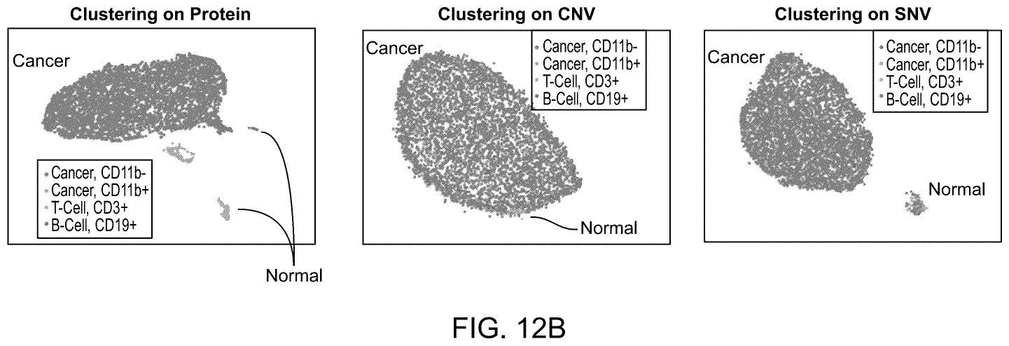

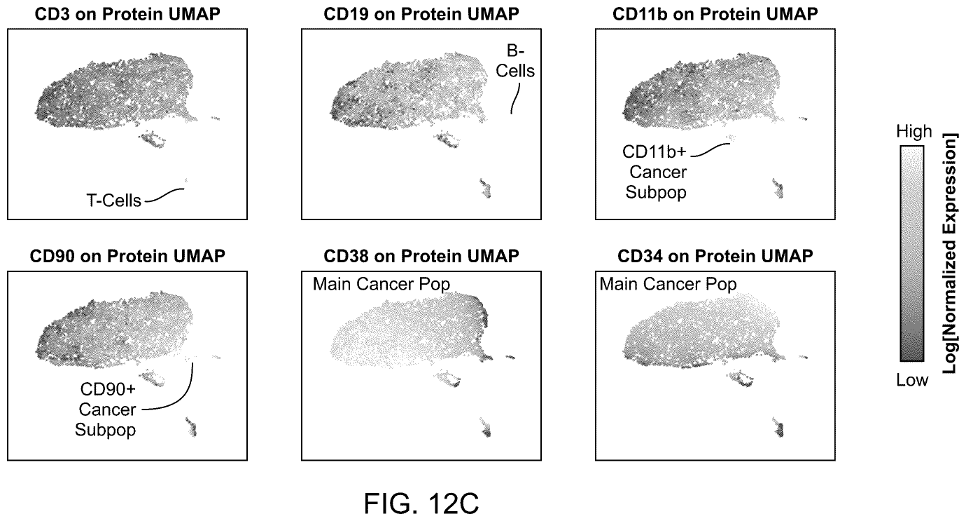

FIGS. 12A-12C illustrate example analysis results of SNV, CNV, and protein of an acute myeloid leukemia (AML) sample. FIG. 12A illustrates an example heatmap of cell types versus SNV, CNV, and surface proteins. FIG. 12B illustrates example clustering on surface proteins, CNV, and SNV. FIG. 12C illustrates results of overlaying individual markers on surface protein clusters (or surface protein expression) based on the protein results as shown in FIG. 12B.

In the methods included in Example 4, the AML sample was analyzed and more than 5000 cells were recovered. The AML sample was found to be 95% cancerous as identified by any of the three analytes. Furthermore, the Chromosome 7 copy loss, a known pathologic mechanism of AML, was detected in CNV and orthogonally validated by loss of heterozygosity in the SNV data, as shown in the heatmap in FIG. 12A. Additionally, the cancerous population was identified by a pathogenic mutation (ASXL1 p.G646V) and higher expression of CD11b, CD34, CD38, and CD90 in two sub populations, as shown in FIGS. 12B and 12C.

### Example 5: Simultaneous Single Cell Analysis Based on Genomic DNA, Intracellular Proteins, and Surface Proteins

In the methods included in Example 5, cells were incubated with barcoded antibodies for surface proteins followed by cell washing to remove unbound antibodies to perform surface and intracellular protein analysis. Cells were then fixed, blocked, and permeabilized before they were incubated with barcoded antibodies targeting intracellular proteins. After washing away the unbound antibodies, cells were introduced into the Tapestri™ instrument where single cells were encapsulated into individual emulsion droplets. Targeted DNA and protein tag amplification occurred in the droplets, and cell identification barcode(s) was added to each amplicon. Protein libraries were separated from the DNA libraries by size and prepared separately.

Utilizing the Tapestri™ platform, concurrent SNV, CNV, surface protein, and intracellular protein analyses were successfully implemented from single cells. The ability to perform a single-cell multi-omic analysis enables researchers to precisely pair DNA data (e.g., information on driver mutations, clone tracing, drug resistant clones, and CNVs) with intracellular protein expression (e.g., information on signal transduction pathways, cell death mechanisms, transcription factors, tumor suppressors, oncoproteins, and drug targeted proteins). The technology may provide a solution to properly couple genotype and phenotype relevant to cancer biology.

### Example 5.1: Analysis of Cell Lines for Detection of Surface Protein and Intracellular Protein

In this example, a mixture of 7 cell lines were analyzed using the Tapestri™ Single-Cell DNA AML Panel, which surveyed 127 amplicons across 20 genes. TotalSeq™-D Heme Oncology Cocktail from BioLegend, which contained 45 antibodies including 3 isotype controls, was used for the surface protein detection. The TotalSeq™-D panel was designed specifically for the Tapestri™ platform. As a proof of concept, 5 intracellular protein targets are included in the experiment.

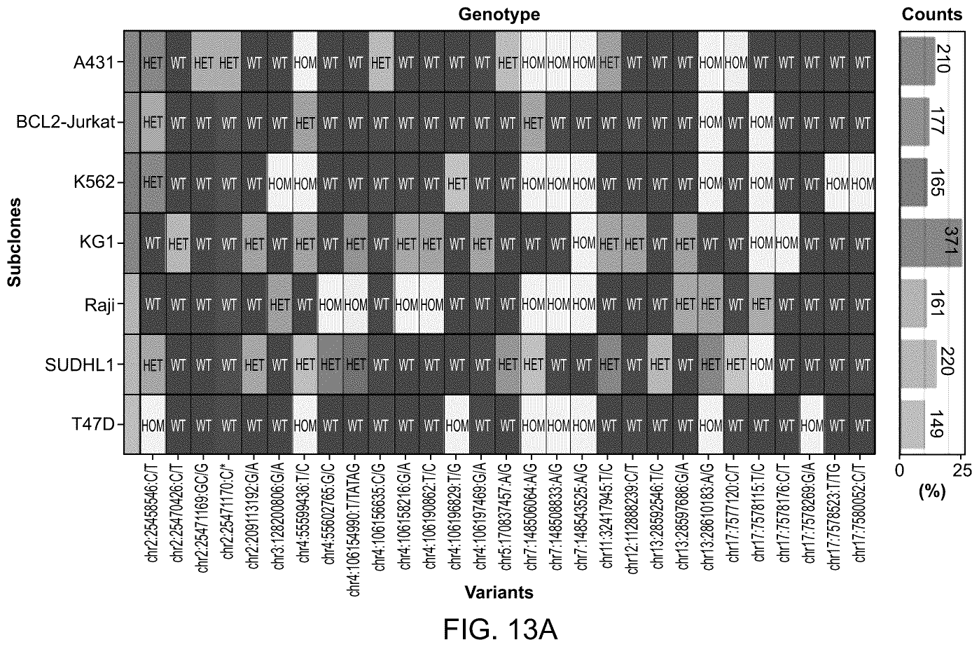

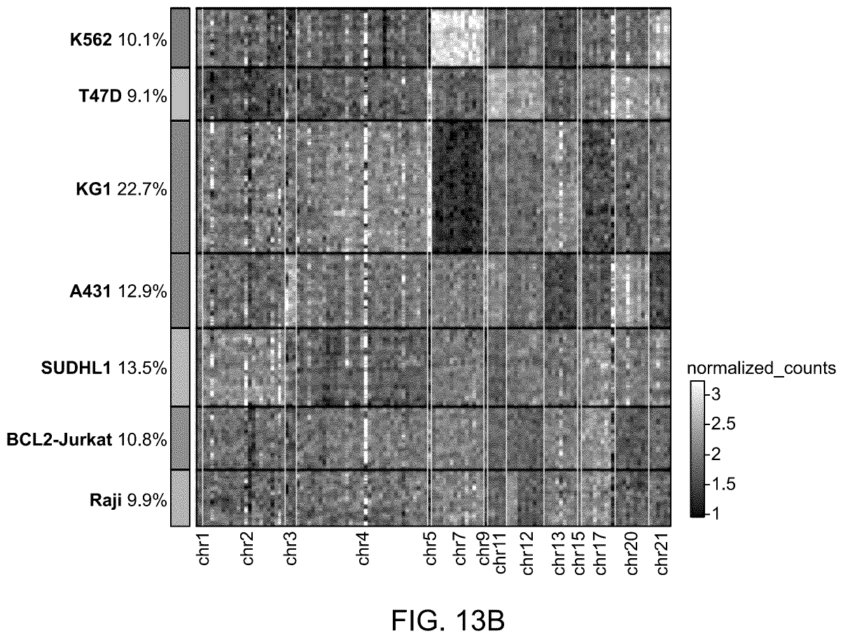

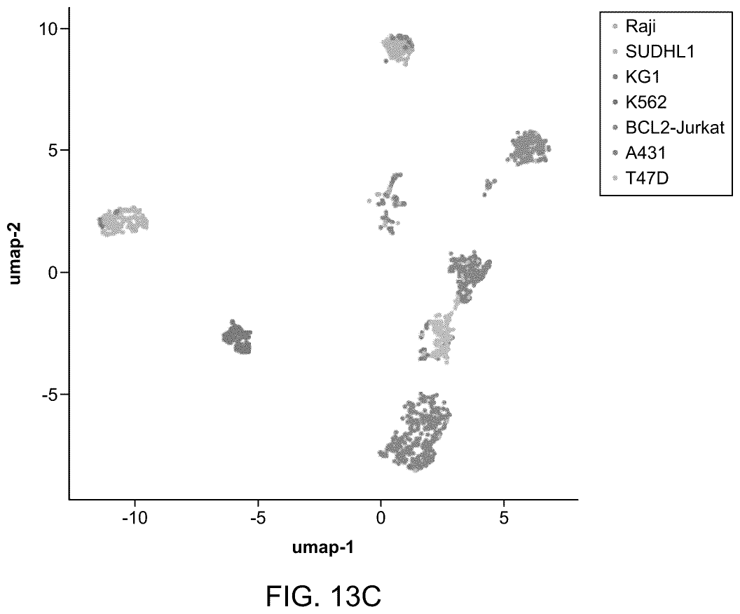

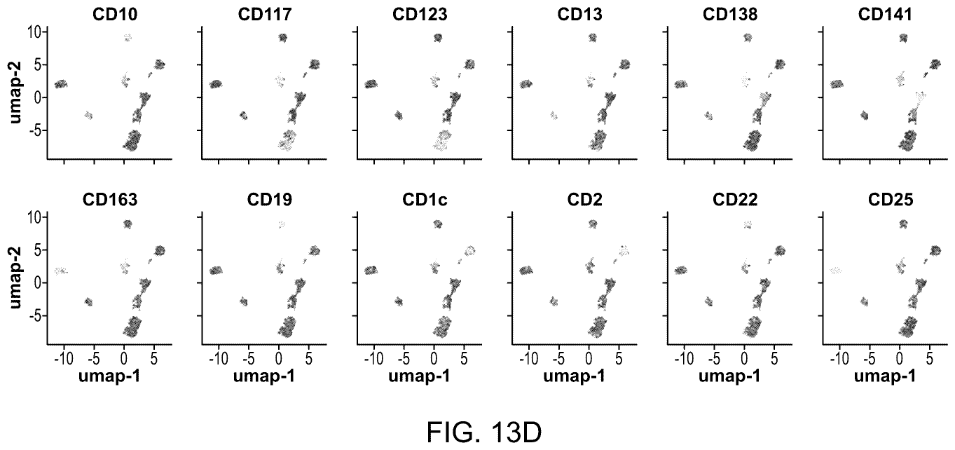

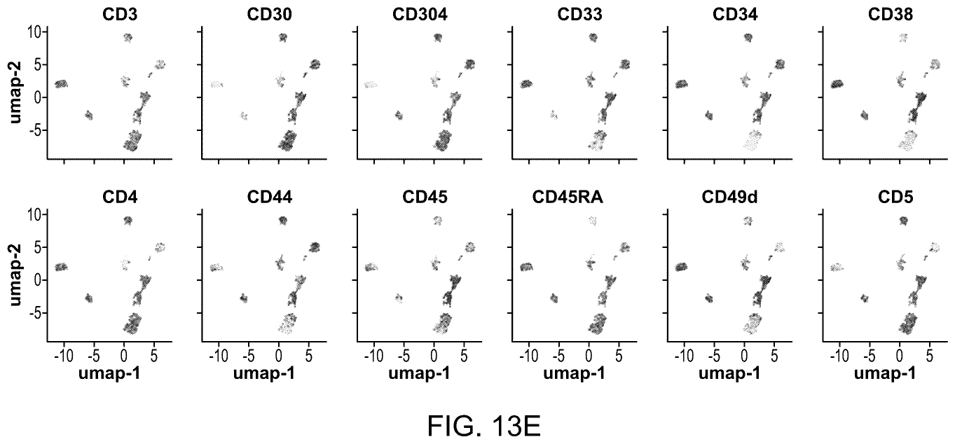

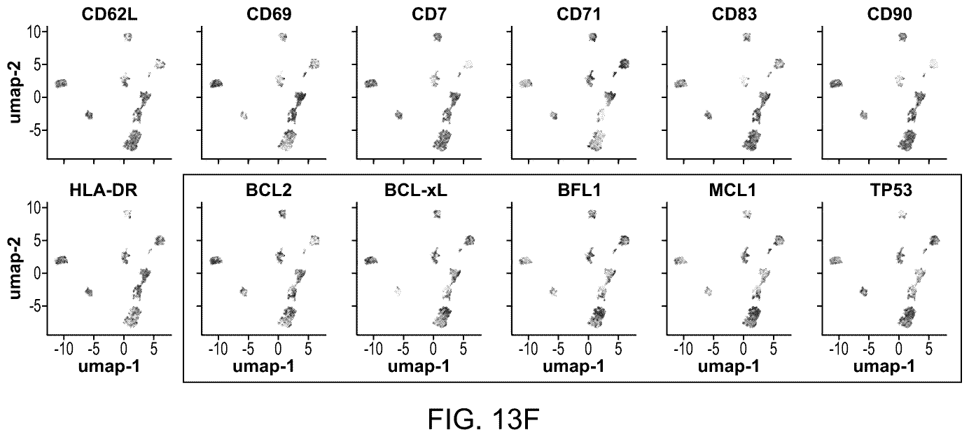

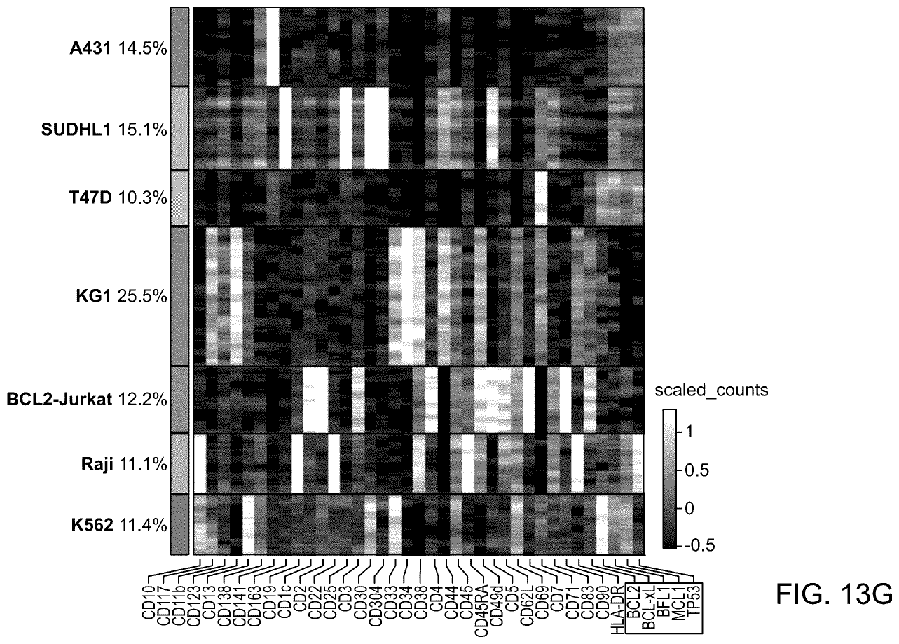

FIG. 13A illustrates example results of cell typing by SNV. FIG. 13B illustrates normalized DNA reads for CNV analysis. FIG. 13C illustrates protein expression uniform manifold approximation and projection (UMAP) by cell type. FIGS. 13D-13F illustrate protein expression UMAP for each protein target. FIG. 13G illustrates a protein expression heatmap.

Using SNV analysis from the DNA data, cells were identified based on their genotypic signatures (FIG. 13A). Normalized read counts from the DNA data were also analyzed to infer CNV information (FIG. 13B). In this example, BCL2-Jurkat was used as the diploid reference for the analysis. The read depth analysis showed that there was a copy number gain in chromosome 7 for K562 cells, and a copy number loss in the same chromosome for KG1 cells.

Furthermore, UMAP analysis was performed on normalized protein reads, and the resulting plot was colored based on the cell type identified by SNV (FIG. 13C). To visualize differential protein expression among cell types, expression profiles were displayed on the UMAP for each protein target (FIGS. 13D-13F), or in a heatmap (FIG. 13G). As shown in FIGS. 13D-13F, 36 out of 50 protein targets were plotted. As shown in FIG. 13G, 37 out of 50 protein targets were plotted. The intracellular protein targets were highlighted in boxes, as shown in FIGS. 13F and 13G. Specifically, FIG. 13F identifies the intracellular proteins of BCL2, BCL-xL, BFL1, MCL1, and TP53. Similarly, FIG. 13G identifies the intracellular proteins of BCL2, BCL-xL, BFL1, MCL1, and TP53.

### Example 5.2: Analysis of Cell Lines Based on Post-Translationally Modified Proteins

To further validate the capability of the method, system, and apparatus as described herein, the workflow was applied on post-translationally modified proteins, including intracellular proteins such as phosphorylated proteins (phospho-ERK, phospho-STAT3) and cleaved proteins (cleaved PARP), other intracellular proteins, and/or surface proteins.

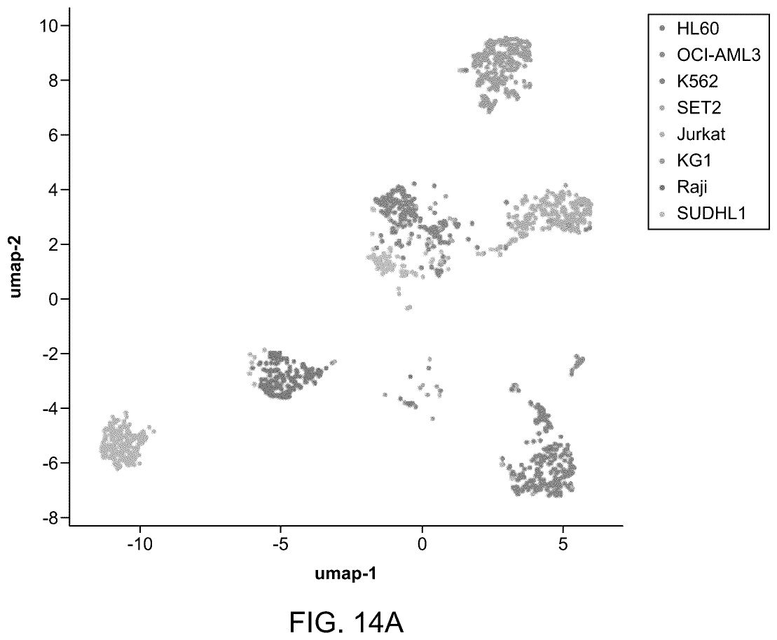

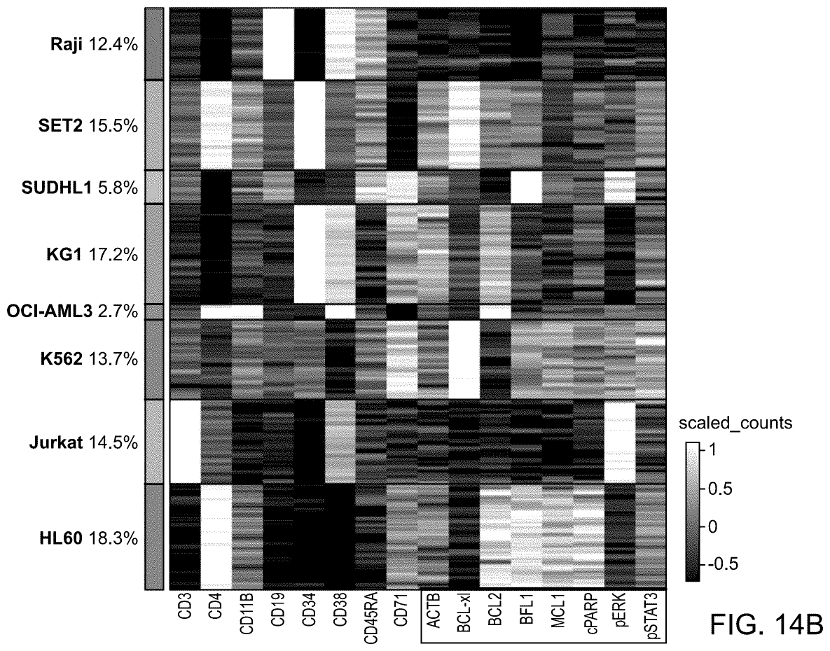

FIGS. 14A and 14B illustrate example results of the workflow applied on phosphorylated proteins (phospho-ERK, phospho-STAT3) and cleaved proteins (cleaved PARP). FIG. 14A illustrate an example protein expression UMAP by cell type. FIG. 14B illustrates an example protein expression heatmap.

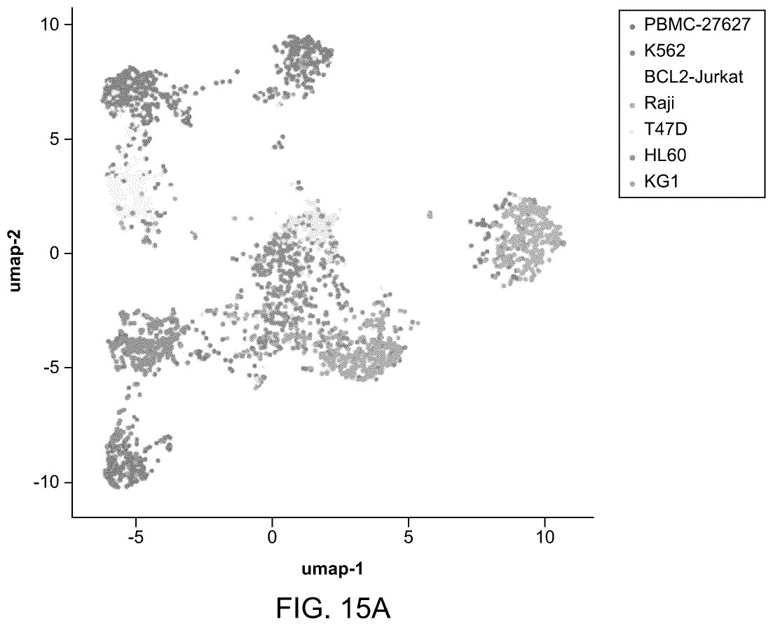

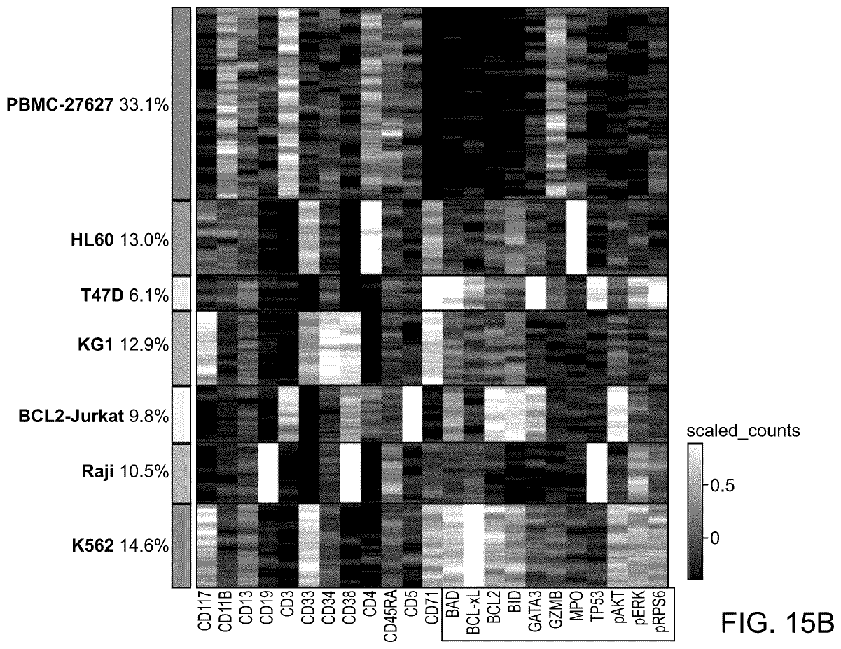

FIGS. 15A and 15B illustrate example results of the workflow applied on peripheral blood mononuclear cell (PMBC) along with cell lines, targeting nuclear proteins (GATA3, TP53) and more phosphorylated proteins (phospho-AKT, phospho-RPS6) than phosphorylated proteins involved in FIGS. 14A and 14B. FIG. 15A illustrate an example protein expression UMAP by cell type. FIG. 15B illustrates an example protein expression heatmap.

As shown in FIGS. 14B and 15B, intracellular protein targets are highlighted in boxes.

### Example 5.3: Multi-Omic Workflow

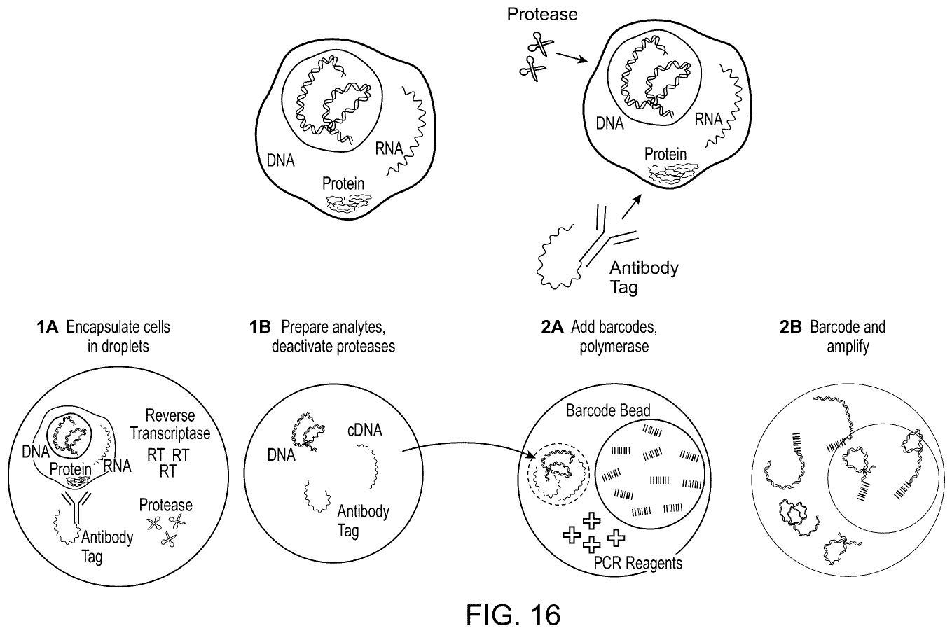

FIG. 16 illustrates a multi-omic workflow including methods utilized in Examples 4 and 5. In general, Examples 4 and 5 utilized the Tapestri™ platform, which applied a multi-omic workflow that is able to simultaneously analyze both genotypic (SNVs, indels, and CNVs) and phenotypic (proteins) factors from thousands of cells individually. This single-cell analysis was performed by a series of analyte-specific processing steps to generate associated DNA sequences. For example, proteins were tagged with oligonucleotide-conjugated antibodies, and gDNA in the nucleus was freed from chromatin via protease digestion. The resulting DNA sequences that were free from nucleus were then be barcoded with a cell-specific sequence for later identification.

In some scenarios, the workflow used in the Examples 4 and 5 enables single-cell DNA and protein detection and included steps that involved two droplets. As shown in FIG. 16, cells were individually encapsulated in a first droplet (e.g., step 1A in FIG. 16, or step 160 in FIG. 1B) for preparing analyte(s). The steps of preparing the analytes(s) included aggressive digestion and/or subsequent protease heat inactivation (e.g., e.g., step 1B in FIG. 16, or step 165 in FIG. 1B). Afterwards, sensitive enzymes that were incompatible with the earlier preparation were added to make a new droplet (e.g., step 2A in FIG. 16, or step 170 in FIG. 1B) where barcoding and amplification proceed (e.g., step 2B in FIG. 16, or step 175 in FIG. 1B). In some scenarios, nuclei were isolated from cells before the workflow and were used throughout the workflow instead of complete cells.

1. A method for analyzing an analyte of a cell, the method comprising:
obtaining a permeabilized cell;
providing an antibody-oligonucleotide conjugate to the permeabilized cell, wherein the antibody-oligonucleotide conjugate enters the permeabilized cell to contact the analyte located internally within the cell to generate an intracellular antibody-oligonucleotide conjugate;
performing a single-cell analysis of the cell, wherein performing the single-cell analysis comprises:
encapsulating the permeabilized cell comprising the intracellular antibody-oligonucleotide conjugate in a droplet;
lysing the permeabilized cell within the droplet to generate a cell lysate comprising the oligonucleotide or a complement of the oligonucleotide;
optionally reverse cross-linking the cell lysate within the droplet using a reducing agent;
re-encapsulating the cell lysate in a second droplet with reagents;
generating amplicons from the oligonucleotide or the complement of the oligonucleotide by performing at least one reaction using the reagents; and
sequencing the amplicons to determine presence or absence of the analyte of the permeabilized cell.

2. The method of claim 1, wherein obtaining a permeabilized cell comprises:
fixing a cell using fixatives;
quenching the fixatives; and
permeabilizing and blocking the cell.

3. The method of claim 1 or 2, wherein providing an antibody-oligonucleotide conjugate to the permeabilized cell comprises:
incubating the permeabilized cell with the antibody-oligonucleotide conjugate; and
washing the permeabilized cell.

4. The method of any one of claims 1-3, wherein providing an antibody-oligonucleotide conjugate to the permeabilized cell comprises:
incubating the permeabilized cell with the antibody-oligonucleotide conjugate for 10 minutes to 30 hours.

5. The method of any one of claims 1-3, wherein providing an antibody-oligonucleotide conjugate to the permeabilized cell comprises:
incubating the permeabilized cell with the antibody-oligonucleotide conjugate for 10-25 hours.

6. The method of any one of claims 1-3, wherein providing an antibody-oligonucleotide conjugate to the permeabilized cell comprises:
incubating the permeabilized cell with the antibody-oligonucleotide conjugate for 16-20 hours.

7. The method of any one of claims 1-3, wherein providing an antibody-oligonucleotide conjugate to the permeabilized cell comprises:
incubating the permeabilized cell with the antibody-oligonucleotide conjugate overnight.

8. The method of any one of claims 1-7, wherein providing an antibody-oligonucleotide conjugate to the permeabilized cell comprises:
incubating the permeabilized cell with the antibody-oligonucleotide conjugate at a temperature between 0-25° C.

9. The method of any one of claims 1-7, wherein providing an antibody-oligonucleotide conjugate to the permeabilized cell comprises:
incubating the permeabilized cell with the antibody-oligonucleotide conjugate at a temperature between 2-8° C.

10. The method of any one of claims 1-7, wherein providing an antibody-oligonucleotide conjugate to the permeabilized cell comprises:
incubating the permeabilized cell with the antibody-oligonucleotide conjugate at a temperature between 3-6° C.

11. The method of any one of claims 1-7, wherein providing an antibody-oligonucleotide conjugate to the permeabilized cell comprises:
incubating the permeabilized cell with the antibody-oligonucleotide conjugate at a temperature of about 4° C.

12. The method of any one of claims 1-11, wherein providing an antibody-oligonucleotide conjugate to the permeabilized cell comprises:
washing the cell for at least 1 minute to wash away unbound antibody-oligonucleotide conjugates.

13. The method of any one of claims 1-11, wherein providing an antibody-oligonucleotide conjugate to the permeabilized cell comprises:
washing the permeabilized cell for at least 3 minutes to wash away unbound antibody-oligonucleotide conjugates.

14. The method of any one of claims 1-11, wherein providing an antibody-oligonucleotide conjugate to the permeabilized cell comprises:
washing the permeabilized cell for at least 5 minutes to wash away unbound antibody-oligonucleotide conjugates.

15. The method of any one of claims 1-14, wherein providing an antibody-oligonucleotide conjugate to the permeabilized cell comprises:
washing the permeabilized cell for one or more times to wash away unbound antibody-oligonucleotide conjugates.

16. The method of any one of claims 1-14, wherein providing an antibody-oligonucleotide conjugate to the permeabilized cell comprises:
washing the permeabilized cell for at least 2 times.

17. The method of any one of claims 1-14, wherein providing an antibody-oligonucleotide conjugate to the permeabilized cell comprises:
washing the permeabilized cell for at least 3 times.

18. The method of any one of claims 1-14, wherein providing an antibody-oligonucleotide conjugate to the permeabilized cell comprises:
washing the permeabilized cell for at least 4 times.

19. A method for analyzing an analyte located on a surface of a cell and an analyte located internally within the cell, the method comprising:
obtaining the cell comprising a surface antibody-oligonucleotide conjugate and an intracellular antibody-oligonucleotide conjugate, the surface antibody-oligonucleotide conjugate being generated by providing a first antibody-oligonucleotide conjugate to be bound to the analyte located on the surface of the cell, and the intracellular antibody-oligonucleotide conjugate being generated by permeabilizing the cell and providing a second antibody-oligonucleotide conjugate to enter the permeabilized cell to contact the analyte located internally within the cell;
performing a single-cell analysis of the cell, wherein performing the single-cell analysis comprises:
encapsulating the permeabilized cell comprising the surface antibody-oligonucleotide conjugate and the intracellular antibody-oligonucleotide conjugate in a first droplet;
lysing the permeabilized cell within the first droplet to generate a cell lysate comprising a first oligonucleotide or a complement of the first oligonucleotide from the first antibody-oligonucleotide conjugate and a second oligonucleotide or a complement of the second oligonucleotide from the second antibody-oligonucleotide conjugate;
optionally, reverse cross-linking the cell lysate within the first droplet using a reducing agent;
re-encapsulating the cell lysate in a second droplet with reagents;
generating first amplicons from the first oligonucleotide or the complement of the first oligonucleotide and second amplicons from the second oligonucleotide or the complement of the second oligonucleotide by performing at least one reaction using the reagents; and
sequencing any one of the first and second amplicons to determine presence or absence of the analyte located on the surface of the cell and the analyte located internally within the permeabilized cell.

20. The method of claim 19, wherein obtaining the cell comprises:
incubating the cell with at least one of the first and the second antibody-oligonucleotide conjugates; and
washing the cell.

21. The method of claim 20, wherein incubating the cell with at least one of the first and the second antibody-oligonucleotide conjugates comprises:
incubating the cell with at least one of the first and the second antibody-oligonucleotide conjugates for 10 minutes to 30 hours.

22. The method of claim 20, wherein incubating the cell with the first and the second antibody-oligonucleotide conjugates comprises:
incubating the cell with the at least one of the first and the second antibody-oligonucleotide conjugates for 10-60 minutes.

23. The method of claim 20, wherein incubating the cell with at least one of the first and the second antibody-oligonucleotide conjugates comprises:
incubating the cell with at least one of the first and the second antibody-oligonucleotide conjugates for 16-20 hours.

24. The method of claim 20, wherein incubating the cell with at least one of the first and the second antibody-oligonucleotide conjugates comprises:
incubating the cell with at least one of the first and the second antibody-oligonucleotide conjugates overnight.

25. The method of any one of claims 20-24, wherein incubating the cell with at least one of the first and the second antibody-oligonucleotide conjugates comprises:
incubating the cell with at least one of the first and the second antibody-oligonucleotide conjugates at a temperature between 0 and 25° C.

26. The method of any one of claims 20-24, wherein incubating the cell with at least one of the first and the second antibody-oligonucleotide conjugates comprises:
incubating the cell with at least one of the first and the second antibody-oligonucleotide conjugates at a temperature between 2 and 25° C.

27. The method of any one of claims 20-24, wherein incubating the cell with at least one of the first and the second antibody-oligonucleotide conjugates comprises:
incubating the cell with at least one of the first and the second antibody-oligonucleotide conjugates on ice.

28. The method of any one of claims 20-24, wherein incubating the cell with at least one of the first and the second antibody-oligonucleotide conjugates comprises:
incubating the cell with at least one of the first and the second antibody-oligonucleotide conjugates at a temperature of about 4° C.

29. The method of any one of claims 20-28, wherein washing the cell comprises:
washing the cell for at least 1 minute to wash away unbound first antibody-oligonucleotide conjugates.

30. The method of any one of claims 20-28, wherein washing the cell comprises:
washing the cell for at least 3 minutes to wash away unbound first antibody-oligonucleotide conjugates.

31. The method of any one of claims 20-28, wherein washing the cell comprises:
washing the cell for at least 5 minutes to wash away unbound first antibody-oligonucleotide conjugates.

32. The method of any one of claims 20-31, wherein washing the cell comprises:
washing the cell for one or more times to wash away unbound first antibody-oligonucleotide conjugates.

33. The method of any one of claims 20-31, wherein washing the cell comprises:
washing the cell for at least 2 times.

34. The method of any one of claims 20-31, wherein washing the cell comprises:
washing the cell for at least 3 times.

35. The method of any one of claims 20-31, wherein washing the cell comprises:
washing the cell for at least 4 times.

36. The method of any one of claims 19-35, further comprising:
providing one or more additional first antibody-oligonucleotide conjugates specific for one or more additional surface analytes to the cell.

37. The method of claim 36, wherein the one or more additional first antibody-oligonucleotide conjugates comprise two additional first antibody-oligonucleotide conjugates specific for two surface analytes.

38. The method of claim 36, wherein the one or more additional first antibody-oligonucleotide conjugates comprise three additional first antibody-oligonucleotide conjugates specific for three surface analytes.

39. The method of claim 36, wherein the one or more additional first antibody-oligonucleotide conjugates comprise four additional first antibody-oligonucleotide conjugates specific for four surface analytes.

40. The method of claim 36, wherein the one or more additional first antibody-oligonucleotide conjugates comprise five additional first antibody-oligonucleotide conjugates specific for five surface analytes.

41. The method of claim 36, wherein the one or more additional first antibody-oligonucleotide conjugates comprise forty-five additional first antibody-oligonucleotide conjugates specific for forty-five surface analytes.

42. The method of any one of claims 19-41, further comprising:
providing one or more additional second antibody-oligonucleotide conjugates specific for one or more additional intracellular analytes to the permeabilized cell.

43. The method of claim 42, wherein the one or more additional second antibody-oligonucleotide conjugates comprise five additional second antibody-oligonucleotide conjugates specific for five intracellular analytes.

44. The method of claim 42, wherein the one or more additional second antibody-oligonucleotide conjugates comprise ten additional second antibody-oligonucleotide conjugates specific for ten intracellular analytes.

45. The method of claim 42, wherein the one or more additional second antibody-oligonucleotide conjugates comprise fifty additional second antibody-oligonucleotide conjugates specific for fifty intracellular analytes.

46. The method of any one of claims 19-45, wherein the second antibody-oligonucleotide conjugate comprises a concentration of up to 13 nM per antibody.

47. The method of any one of claims 1-46, wherein obtaining a cell comprises:
fixing the cell using fixatives for at least 30 minutes.

48. The method of any one of claims 1-46, wherein obtaining a cell comprises:
fixing the cell using fixatives for at least 45 minutes.

49. The method of any one of claims 1-46, wherein obtaining a cell comprises:
fixing the cell using fixatives for at least 60 minutes.

50. The method of any one of claims 1-46, wherein obtaining a cell comprises:
fixing the cell using fixatives for at least 90 minutes.

51. The method of any one of claims 1-50, wherein obtaining a cell comprises:
fixing the cell using fixatives at a temperature between 4 and 50° C.

52. The method of any one of claims 1-50, wherein obtaining a cell comprises:
fixing the cell using fixatives at a temperature between 10 and 30° C.

53. The method of any one of claims 1-50, wherein obtaining a cell comprises:
fixing the cell using fixatives at a temperature between 20 and 25° C.

54. The method of any one of claims 1-53, wherein obtaining a cell comprises:
fixing the cell using 0.1 mM to 20 mM of one or more fixatives in a reactive volume using a background buffer.

55. The method of any one of claims 1-53, wherein obtaining a cell comprises:
fixing the cell using 0.5 mM to 10 mM of one or more fixatives in a reactive volume using a background buffer.

56. The method of any one of claims 1-53, wherein obtaining a cell comprises:
fixing the cell using 1 mM to 5 mM of one or more fixatives in a reactive volume using a background buffer.

57. The method of any one of claims 54-56, wherein the reactive volume is from 0.01 to 10 mL.

58. The method of any one of claims 54-56, wherein the reactive volume is from 0.05 to 5 mL.

59. The method of any one of claims 54-56, wherein the reactive volume is from 0.1 to 1 mL.

60. The method of any one of claims 54-59, wherein the background buffer is Dulbecco's phosphate-buffered saline (DPBS).

61. The method of any one of claims 1-60, wherein obtaining a cell comprises:
quenching the cell for at least 1 minute.

62. The method of any one of claims 1-60, wherein obtaining a cell comprises:
quenching the cell for at least 5 minutes.

63. The method of any one of claims 1-60, wherein obtaining a cell comprises:
quenching the cell for at least 10 minutes.

64. The method of any one of claims 1-63, wherein obtaining a cell comprises:
quenching the cell at a temperature between 10 and 50° C.

65. The method of any one of claims 1-63, wherein obtaining a cell comprises:
quenching the cell at a temperature between 10 and 30° C.

66. The method of any one of claims 1-63, wherein obtaining a cell comprises:
quenching the cell at a temperature between 20 and 25° C.

67. The method of any one of claims 1-66, wherein obtaining a cell comprises:
permeabilizing and blocking the cell for at least 10 minutes.

68. The method of any one of claims 1-66, wherein obtaining a cell comprises:
permeabilizing and blocking the cell for at least 20 minutes.

69. The method of any one of claims 1-66, wherein obtaining a cell comprises:
permeabilizing and blocking the cell for at least 30 minutes.

70. The method of any one of claims 1-69, wherein obtaining a cell comprises:
permeabilizing and blocking the cell at a temperature between 10 and 50° C.

71. The method of any one of claims 1-69, wherein obtaining a cell comprises:
permeabilizing and blocking the cell at a temperature between 10 and 30° C.

72. The method of any one of claims 1-69, wherein obtaining a cell comprises:
permeabilizing and blocking the cell at a temperature between 20 and 25° C.

73. The method of any one of claims 1-72, where the single-cell analysis is performed for at least 1 million cells in one workflow.

74. The method of any one of claims 1-73, wherein the reagents comprise one or more antibody tag primers.

75. The method of any one of claims 1-74, wherein the one or more antibody tag primers comprises at least 10 primer reagents.

76. The method of any one of claims 1-75, wherein the one or more antibody tag primers comprises at least 50 primer reagents.

77. The method of any one of claims 1-76, wherein the one or more antibody tag primers comprises at least 100 primer reagents.

78. The method of any one of claims 1-77, wherein the one or more antibody tag primers comprises at least 150 primer reagents.

79. The method of any one of claims 1-78, wherein the reagents comprise one or more barcodes in the second droplet.

80. The method of any one of claims 1-79, wherein the reagents comprise polymerase.

81. The method of any one of claims 1-80, wherein the at least one reaction comprises nucleic acid amplification.

82. The method of any one of claims 1-81, wherein the at least one reaction comprises polymerase chain reaction (PCR).

83. The method of any one of claims 1-82, wherein the at least one reaction comprises loop-mediated isothermal amplification (LAMP).

84. The method of any one of claims 1-83, wherein the cell lysate comprises genomic DNA.

85. The method of claim 84, further comprising generating amplicons from the genomic DNA.

86. The method of claim 85, further comprising sequencing the amplicons generated from the genomic DNA.

87. The method of claim 86, further comprising determining presence or absence of one or more mutations based on the sequenced amplicons generated from the genomic DNA.

88. The method of claim 87, wherein the one or more mutations comprise any one of single-nucleotide polymorphism (SNV), insertion or deletion mutation (indel), or copy number variation (CNV).

89. The method of any one of claims 19-88, wherein permeabilizing the cell comprises permeabilizing the cell using a permeabilization buffer, and wherein the permeabilization buffer comprises at least one of Triton™ X-100, Prionex® gelatin, salmon sperm DNA, mouse IgG, and EDTA.

90. The method of any one of claims 19-88, wherein permeabilizing the cell comprises permeabilizing the cell using a permeabilization buffer, wherein the permeabilization buffer comprises a 0.1% solution.

91. The method of any one of claims 1-90, wherein lysing the permeabilized cell within the droplet comprises:
optionally applying extra reagents, wherein the amount of the extra reagents is less than 1 mM DTT.

92. The method of any one of claims 1-90, wherein lysing the permeabilized cell within the droplet comprises:
optionally applying extra reagents, wherein the amount of the extra reagents is less than 2 mM DTT.

93. The method of any one of claims 1-90, wherein lysing the permeabilized cell within the droplet comprises:
optionally applying extra reagents, wherein the amount of the extra reagents is less than 5M DTT.

94. The method of any one of claims 1-93, wherein the method is applied to one or more cell lines.

95. The method of any one of claims 1-94, wherein the analyte is at least one of surface protein and intracellular protein.

96. The method of any one of claims 1-95, wherein the cell lysate comprises RNA.

97. The method of claim 96, further comprising reverse transcribing the RNA to generate cDNA, and generating amplicons from the cDNA.

98. The method of any one of claim 97, further comprising sequencing the amplicons generated from the cDNA.

99. A method for analyzing an analyte of a cell, the method comprising:
obtaining a cell nucleus isolated from the cell;
providing an antibody-oligonucleotide conjugate to the cell nucleus, wherein the antibody-oligonucleotide conjugate contacts the analyte of the cell nucleus to generate an intracellular antibody-oligonucleotide conjugate;
performing a single-cell analysis of the cell, wherein performing the single-cell analysis comprises:
encapsulating the cell nucleus comprising the intracellular antibody-oligonucleotide conjugate in a droplet;
generating a cell nucleus lysate within the droplet comprising the oligonucleotide or a complement of the oligonucleotide;
optionally reverse cross-linking the cell nucleus lysate within the droplet using a reducing agent;
re-encapsulating the cell lysate in a second droplet with reagents;
generating amplicons from the oligonucleotide or the complement of the oligonucleotide by performing at least one reaction using the reagents; and
sequencing the amplicons to determine presence or absence of the analyte of the cell nucleus.

100. The method of claim 99, wherein obtaining the cell nucleus isolated from the cell comprises:
incubating the cell nucleus with the antibody-oligonucleotide conjugate; and
washing the cell nucleus.

101. The method of claim 99 or 100, further comprising:
providing one or more additional antibody-oligonucleotide conjugates specific for one or more additional analytes to the cell nucleus.

102. The method of any one of claims 99-101, wherein the cell nucleus lysate comprises genomic DNA.

103. The method of claim 102, further comprising generating amplicons from the genomic DNA.

104. The method of claim 103, further comprising sequencing the amplicons generated from the genomic DNA.

105. The method of claim 104, further comprising determining presence or absence of one or more mutations based on the sequenced amplicons generated from the genomic DNA.

106. The method of claim 105, wherein the one or more mutations comprise any one of single-nucleotide polymorphism (SNV), insertion or deletion mutation (indel), or copy number variation (CNV).
